SEQUENCE LISTING (149 sequences)

SEQ ID NO: 1-6 - skipped (6 entries)

SEQ ID NO: 7 - DNA, 4015 bp, Homo sapiens
gcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 8 - DNA, 451 bp, Homo sapiens
gggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctgctggcccgttcgcccctcccggggacctgcggcgggtcgcctgcccagcccccgaaccccgcctggaggccgcggtcggcccggggcttctccggaggcacccactgccaccgcgaagagttgggctctgtcagccgcgggtctctcgggggcgagggcgaggttcaggcctttcaggccgcaggaagaggaacggagcgagtccccgcgcgcggcgcgattccctgagctgtgggacgtgcacccaggactcggctcacacatgc

SEQ ID NO: 9 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 10 - skipped

SEQ ID NO: 11 - DNA, 22 bp, Artificial Sequence
  Description of Artificial Sequence: Synthetic primer
gagtttcaggcagcgctgcgtc

SEQ ID NO: 12 - DNA, 23 bp, Artificial Sequence
  Description of Artificial Sequence: Synthetic primer
caggacgcgtggaccgagtgacc

SEQ ID NO: 13 - skipped

SEQ ID NO: 14 - DNA, 23 bp, Artificial Sequence
  Description of Artificial Sequence: Synthetic primer
cttggtgagctggatgtgcggtg

SEQ ID NO: 15 - DNA, 23 bp, Artificial Sequence
  Description of Artificial Sequence: Synthetic primer
gcctcctgaggggctctctattg

SEQ ID NO: 16 - DNA, 23 bp, Artificial Sequence
  Description of Artificial Sequence: Synthetic primer
gccggatccactttcctgactgt

SEQ ID NO: 17 - DNA, 22 bp, Artificial Sequence
  Description of Artificial Sequence: Synthetic primer
gtctgatgtgtggtagctgcag

SEQ ID NO: 18 - DNA, 23 bp, Artificial Sequence
  Description of Artificial Sequence: Synthetic primer
gtggtcctgcctgtctcagcacc

SEQ ID NO: 19 - DNA, 21 bp, Artificial Sequence
  Description of Artificial Sequence: Synthetic primer
ggctgaatggtagacgtgtcg

SEQ ID NO: 20 - DNA, 23 bp, Artificial Sequence
  Description of Artificial Sequence: Synthetic primer
gcacagaattgcacaagctgatg

SEQ ID NO: 21 - DNA, 22 bp, Artificial Sequence
  Description of Artificial Sequence: Synthetic primer
gcatcctggggctgacattgcc

SEQ ID NO: 22 - DNA, 22 bp, Artificial Sequence
  Description of Artificial Sequence: Synthetic primer
gtccatggagtgagcacccagc

SEQ ID NO: 23 - DNA, 23 bp, Artificial Sequence
  Description of Artificial Sequence: Synthetic primer
gatgacacagagtcttgactcgc

SEQ ID NO: 24 - DNA, 21 bp, Artificial Sequence
  Description of Artificial Sequence: Synthetic primer
cacacccacgagcaccgtctg

SEQ ID NO: 25 - DNA, 21 bp, Artificial Sequence
  Description of Artificial Sequence: Synthetic primer
tgtgactcctcagcctctgtt

SEQ ID NO: 26 - DNA, 18 bp, Artificial Sequence
  Description of Artificial Sequence: Synthetic primer
gccgctccagcgtcactg

SEQ ID NO: 27 - DNA, 23 bp, Artificial Sequence
  Description of Artificial Sequence: Synthetic primer
caggtgaaccagcacgtcgtcgc

SEQ ID NO: 28 - DNA, 21 bp, Artificial Sequence
  Description of Artificial Sequence: Synthetic primer
cttgtcgcctgaggagtagag

SEQ ID NO: 29 - DNA, 21 bp, Artificial Sequence
  Description of Artificial Sequence: Synthetic primer
gtgaacctcgtaagtttatgc

SEQ ID NO: 30 - DNA, 23 bp, Artificial Sequence
  Description of Artificial Sequence: Synthetic primer
gagccttgcacagaatccacttg

SEQ ID NO: 31 - DNA, 23 bp, Artificial Sequence
  Description of Artificial Sequence: Synthetic primer
ctcccaaggtccagcagggctgc

SEQ ID NO: 32 - DNA, 24 bp, Artificial Sequence
  Description of Artificial Sequence: Synthetic primer
cacagacacgactgcattctagac

SEQ ID NO: 33 - DNA, 22 bp, Artificial Sequence
  Description of Artificial Sequence: Synthetic primer
caacagtctgtccggtcatgag

SEQ ID NO: 34 - DNA, 23 bp, Artificial Sequence
  Description of Artificial Sequence: Synthetic primer
gcttgccatttccaggcctcgtg

SEQ ID NO: 35 - DNA, 22 bp, Artificial Sequence
  Description of Artificial Sequence: Synthetic primer
gagcagtcatggtctccagagc

SEQ ID NO: 36 - DNA, 22 bp, Artificial Sequence
  Description of Artificial Sequence: Synthetic primer
ctgctcttgcggatccagcacc

SEQ ID NO: 37 - DNA, 21 bp, Artificial Sequence
  Description of Artificial Sequence: Synthetic primer
cagccgggcacaggctccact

SEQ ID NO: 38 - DNA, 21 bp, Artificial Sequence
  Description of Artificial Sequence: Synthetic primer
cagtcaccatcagccttgcag

SEQ ID NO: 39 - DNA, 21 bp, Artificial Sequence
  Description of Artificial Sequence: Synthetic primer
cgcgaacagaactgtgcacag

SEQ ID NO: 40 - DNA, 21 bp, Artificial Sequence
  Description of Artificial Sequence: Synthetic primer
acagggcgttcaaggatgacc

SEQ ID NO: 41 - DNA, 22 bp, Artificial Sequence
  Description of Artificial Sequence: Synthetic primer
cagcagcatctgaggctgctcg

SEQ ID NO: 42 - DNA, 18 bp, Artificial Sequence
  Description of Artificial Sequence: Synthetic primer
agtgcacattaggattca

SEQ ID NO: 43 - DNA, 23 bp, Artificial Sequence
  Description of Artificial Sequence: Synthetic primer
gaggtttcaggcagcgctgcgtc

SEQ ID NO: 44 - DNA, 23 bp, Artificial Sequence
  Description of Artificial Sequence: Synthetic primer
caggtgaaccagcacgtcgtcgc

SEQ ID NO: 45 - DNA, 22 bp, Artificial Sequence
  Description of Artificial Sequence: Synthetic primer
caacacggtgaccgacgcactg

SEQ ID NO: 46 - DNA, 21 bp, Artificial Sequence
  Description of Artificial Sequence: Synthetic primer
cttgtcgcctgaggagtagag

SEQ ID NO: 47 - DNA, 24 bp, Artificial Sequence
  Description of Artificial Sequence: Synthetic primer
caggacggcgtggaccgagtgacc

SEQ ID NO: 48 - DNA, 21 bp, Artificial Sequence
  Description of Artificial Sequence: Synthetic primer
gtgaacctcgtaagtttatgc

SEQ ID NO: 49 - DNA, 23 bp, Artificial Sequence
  Description of Artificial Sequence: Synthetic primer
cttggtgagctggatgtgcggtg

SEQ ID NO: 50 - DNA, 23 bp, Artificial Sequence
  Description of Artificial Sequence: Synthetic primer
gagccttgcacagaatccacttg

SEQ ID NO: 51 - DNA, 23 bp, Artificial Sequence
  Description of Artificial Sequence: Synthetic primer
gcctcctgaggggctctctattg

SEQ ID NO: 52 - DNA, 23 bp, Artificial Sequence
  Description of Artificial Sequence: Synthetic primer
ctcccaaggtccagcagggctgc

SEQ ID NO: 53 - DNA, 23 bp, Artificial Sequence
  Description of Artificial Sequence: Synthetic primer
gccggatccactttcctgactgt

SEQ ID NO: 54 - DNA, 24 bp, Artificial Sequence
  Description of Artificial Sequence: Synthetic primer
cacagacacgactgcattctagac

SEQ ID NO: 55 - DNA, 22 bp, Artificial Sequence
  Description of Artificial Sequence: Synthetic primer
gtctgatgtgtggtagctgcag

SEQ ID NO: 56 - DNA, 22 bp, Artificial Sequence
  Description of Artificial Sequence: Synthetic primer
caacagtctgtccggtcatgag

SEQ ID NO: 57 - DNA, 23 bp, Artificial Sequence
  Description of Artificial Sequence: Synthetic primer
gtggtcctgcctgtctcagcacc

SEQ ID NO: 58 - DNA, 23 bp, Artificial Sequence
  Description of Artificial Sequence: Synthetic primer
gcttgccatttccaggcctcgtg

SEQ ID NO: 59 - DNA, 21 bp, Artificial Sequence
  Description of Artificial Sequence: Synthetic primer
ggctgaatggtagacgtgtcg

SEQ ID NO: 60 - DNA, 22 bp, Artificial Sequence
  Description of Artificial Sequence: Synthetic primer
gagcagtcatggtctccagagc

SEQ ID NO: 61 - DNA, 23 bp, Artificial Sequence
  Description of Artificial Sequence: Synthetic primer
gcacagaattgcacaagctgatg

SEQ ID NO: 62 - DNA, 22 bp, Artificial Sequence
  Description of Artificial Sequence: Synthetic primer
ctgctcttgcggatccagcacc

SEQ ID NO: 63 - DNA, 22 bp, Artificial Sequence
  Description of Artificial Sequence: Synthetic primer
gcatcctggggctgacattgcc

SEQ ID NO: 64 - DNA, 21 bp, Artificial Sequence
  Description of Artificial Sequence: Synthetic primer
cagccgggcacaggctccact

SEQ ID NO: 65 - DNA, 22 bp, Artificial Sequence
  Description of Artificial Sequence: Synthetic primer
gtccatggagtgagcacccagc

SEQ ID NO: 66 - DNA, 21 bp, Artificial Sequence
  Description of Artificial Sequence: Synthetic primer
cagtcaccatcagccttgcag

SEQ ID NO: 67 - DNA, 23 bp, Artificial Sequence
  Description of Artificial Sequence: Synthetic primer
gatgacacagagtcttgactcgc

SEQ ID NO: 68 - DNA, 21 bp, Artificial Sequence
  Description of Artificial Sequence: Synthetic primer
cgcgaacagaactgtgcacag

SEQ ID NO: 69 - DNA, 21 bp, Artificial Sequence
  Description of Artificial Sequence: Synthetic primer
cacacccacgagcaccgtctg

SEQ ID NO: 70 - DNA, 21 bp, Artificial Sequence
  Description of Artificial Sequence: Synthetic primer
acagggcgttcaaggatgacc

SEQ ID NO: 71 - DNA, 21 bp, Artificial Sequence
  Description of Artificial Sequence: Synthetic primer
tgtgactcctcagcctctgtt

SEQ ID NO: 72 - DNA, 22 bp, Artificial Sequence
  Description of Artificial Sequence: Synthetic primer
cagcagcatctgaggctgctcg

SEQ ID NO: 73 - DNA, 18 bp, Artificial Sequence
  Description of Artificial Sequence: Synthetic primer
gccgctccagcgtcactg

SEQ ID NO: 74 - DNA, 18 bp, Artificial Sequence
  Description of Artificial Sequence: Synthetic primer
agtgcacattaggattca

SEQ ID NO: 75 - DNA, 10 bp, Homo sapiens
ccgaagaagc

SEQ ID NO: 76 - DNA, 10 bp, Homo sapiens
ggccccccat

SEQ ID NO: 77 - DNA, 10 bp, Homo sapiens
ggtgtctgtg

SEQ ID NO: 78 - DNA, 10 bp, Homo sapiens
ctgaatgcag

SEQ ID NO: 79 - DNA, 10 bp, Homo sapiens
gtcacaggcc

SEQ ID NO: 80 - DNA, 10 bp, Homo sapiens
gcctggtcca

SEQ ID NO: 81 - DNA, 10 bp, Homo sapiens
gaggtcaggc

SEQ ID NO: 82 - DNA, 10 bp, Homo sapiens
tttggtttaa

SEQ ID NO: 83 - DNA, 10 bp, Homo sapiens
gtgaggtggc

SEQ ID NO: 84 - DNA, 10 bp, Homo sapiens
gggccrgggc

SEQ ID NO: 85 - DNA, 10 bp, Homo sapiens
gggccrgggc

SEQ ID NO: 86 - DNA, 10 bp, Homo sapiens
tggcatgagg

SEQ ID NO: 87 - DNA, 10 bp, Homo sapiens
ggtctgggtg

SEQ ID NO: 88 - DNA, 10 bp, Homo sapiens
gcctctgtgc

SEQ ID NO: 89 - DNA, 10 bp, Homo sapiens
gccgctgtac

SEQ ID NO: 90 - DNA, 10 bp, Homo sapiens
ccgcctgagc

SEQ ID NO: 91 - DNA, 10 bp, Homo sapiens
tggggccgac

SEQ ID NO: 92 - DNA, 10 bp, Homo sapiens
cccccgtttc

SEQ ID NO: 93 - DNA, 10 bp, Homo sapiens
gtttccgaaa

SEQ ID NO: 94 - DNA, 10 bp, Homo sapiens
gctttggaga

SEQ ID NO: 95 - DNA, 10 bp, Homo sapiens
gttcagagtt

SEQ ID NO: 96 - DNA, 10 bp, Homo sapiens
crcgcgtgcc

SEQ ID NO: 97 - DNA, 10 bp, Homo sapiens
cgcgtgcctg

SEQ ID NO: 98 - DNA, 10 bp, Homo sapiens
gcgctctgct

SEQ ID NO: 99 - DNA, 10 bp, Homo sapiens
ctgtgcacag

SEQ ID NO: 100 - DNA, 10 bp, Homo sapiens
gcatgtgtgc

SEQ ID NO: 101 - DNA, 10 bp, Homo sapiens
cccgcagacc

SEQ ID NO: 102 - DNA, 10 bp, Homo sapiens
ccggccctst

SEQ ID NO: 103 - DNA, 10 bp, Homo sapiens
tgccctccga

SEQ ID NO: 104 - DNA, 10 bp, Homo sapiens
gcccccgggc

SEQ ID NO: 105 - DNA, 10 bp, Homo sapiens
ggaagctccc

SEQ ID NO: 106 - DNA, 11 bp, Homo sapiens
gcaccgctggg

SEQ ID NO: 107 - DNA, 10 bp, Homo sapiens
ccgaagaagc

SEQ ID NO: 108 - DNA, 10 bp, Homo sapiens
ggccccccat

SEQ ID NO: 109 - DNA, 10 bp, Homo sapiens
actgcccgct

SEQ ID NO: 110 - DNA, 10 bp, Homo sapiens
ctgaatgcag

SEQ ID NO: 111 - DNA, 10 bp, Homo sapiens
ccaggccctt

SEQ ID NO: 112 - DNA, 10 bp, Homo sapiens
gtcacaggcc

SEQ ID NO: 113 - DNA, 10 bp, Homo sapiens
gcctggtcca

SEQ ID NO: 114 - DNA, 10 bp, Homo sapiens
tttggtttaa

SEQ ID NO: 115 - DNA, 10 bp, Homo sapiens
tttaacttcc

SEQ ID NO: 116 - DNA, 10 bp, Homo sapiens
ggcccggagg

SEQ ID NO: 117 - DNA, 10 bp, Homo sapiens
gcacrgtgag

SEQ ID NO: 118 - DNA, 10 bp, Homo sapiens
gtgaggtggc

SEQ ID NO: 119 - DNA, 10 bp, Homo sapiens
gggccrgggc

SEQ ID NO: 120 - DNA, 10 bp, Homo sapiens
ctccgtgcgc

SEQ ID NO: 121 - DNA, 10 bp, Homo sapiens
tggcatgagg

SEQ ID NO: 122 - DNA, 10 bp, Homo sapiens
ggtctgggtg

SEQ ID NO: 123 - DNA, 10 bp, Homo sapiens
ggatccactt

SEQ ID NO: 124 - DNA, 10 bp, Homo sapiens
gcctctgtgc

SEQ ID NO: 125 - DNA, 10 bp, Homo sapiens
caggacccgc

SEQ ID NO: 126 - DNA, 10 bp, Homo sapiens
tggggccgac

SEQ ID NO: 127 - DNA, 10 bp, Homo sapiens
ccaggccagg

SEQ ID NO: 128 - DNA, 10 bp, Homo sapiens
grcccccgtt

SEQ ID NO: 129 - DNA, 10 bp, Homo sapiens
gtggctcacc

SEQ ID NO: 130 - DNA, 10 bp, Homo sapiens
gcgtccacct

SEQ ID NO: 131 - DNA, 10 bp, Homo sapiens
gcaagtatgt

SEQ ID NO: 132 - DNA, 10 bp, Homo sapiens
crcgcgtgcc

SEQ ID NO: 133 - DNA, 10 bp, Homo sapiens
cgcgtgcctg

SEQ ID NO: 134 - DNA, 10 bp, Homo sapiens
ctgtgcacag

SEQ ID NO: 135 - DNA, 10 bp, Homo sapiens
tgcacgcaca

SEQ ID NO: 136 - DNA, 10 bp, Homo sapiens
gcatgtgtgc

SEQ ID NO: 137 - DNA, 10 bp, Homo sapiens
atctctgaca

SEQ ID NO: 138 - DNA, 10 bp, Homo sapiens
tgtgcaggtg

SEQ ID NO: 139 - DNA, 10 bp, Homo sapiens
ccggccctst

SEQ ID NO: 140 - DNA, 10 bp, Homo sapiens
ctgaccctgg

SEQ ID NO: 141 - DNA, 10 bp, Homo sapiens
gggacgacgc

SEQ ID NO: 142 - DNA, 11 bp, Homo sapiens
gcaccgctggg

SEQ ID NO: 143 - DNA, 10 bp, Homo sapiens
gctgaaggct

SEQ ID NO: 144 - DNA, 10 bp, Artificial Sequence
  Description of Artificial Sequence: Synthetic oligonucleotide
tgcgtggtgt

SEQ ID NO: 145 - DNA, 10 bp, Artificial Sequence
  Description of Artificial Sequence: Synthetic oligonucleotide
ccagatgggc

SEQ ID NO: 146 - DNA, 10 bp, Artificial Sequence
  Description of Artificial Sequence: Synthetic oligonucleotide
gggcccgggc

SEQ ID NO: 147 - DNA, 10 bp, Artificial Sequence
  Description of Artificial Sequence: Synthetic oligonucleotide
ttttcggctg

SEQ ID NO: 148 - DNA, 10 bp, Artificial Sequence
  Description of Artificial Sequence: Synthetic oligonucleotide
gggacgacgc

SEQ ID NO: 149 - DNA, 10 bp, Artificial Sequence
  Description of Artificial Sequence: Synthetic oligonucleotide
cgctgttttt

(19) United States
(12) Patent Application Publication
(10) Pub. No.: US 2010/0291560 A1
(43) Pub. Date: Nov. 18, 2010

(54) METHODS AND COMPOSITIONS FOR DIAGNOSIS AND TREATMENT OF DYSKERATOSIS CONGENITA AND RELATED DISORDERS

(75) Inventor: Mary Y. Armanios, Ellicott City, MD (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 12/593,500

(22) PCT Filed: Mar. 27, 2008
(86) PCT No.: PCT/US08/58455

§ 371 Date: Jul. 30, 2010

Related U.S. Application Data
(60) Provisional application No. 60/920,252, filed on Mar. 27, 2007.

(51) Int. Cl. C12Q 1/68; C07H 21/04; G01N 33/53

(52) U.S. Cl. 435/6; 536/24.33; 436/94; 436/501

(57) ABSTRACT

The present invention features methods of detection, diagnosing a presence or a predisposition to, or determining the risk for a subject to develop telomere-associated diseases or disorders. The methods include detecting the presence or absence of an alteration in a nucleic acid in a sample. The methods also include determining telomere length. In certain cases, average telomere length is a surrogate marker for a telomere-associated disease or disorder.

### PRIORITY STATEMENT

This application claims priority to U.S. Provisional Application 60/920,252, filed on Mar. 27, 2007, and incorporated by reference in its entirety herein.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services. This research was supported by grant NCI K08118416 from the National Institute of Health. The Government may have certain rights in this invention.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Idiopathic Pulmonary Fibrosis has a predictable, progressive clinical course that ultimately leads to respiratory failure. Irreversible fibrosis is the hallmark, which has a characteristic radiographic appearance most often associated with the pathological lesion of usual interstitial pneumonia. Although both genetic and environmental factors have been implicated, the cause of idiopathic pulmonary fibrosis is unknown, and treatment approaches that target immune system have not proved to be successful.

From 2 to 20% of patients with idiopathic pulmonary fibrosis have a family history of disease; inheritance appears to be autosomal dominant with variable penetrance. Aside from the identification of large kindred with a mutation in the gene surfactant protein C in affected family members, the genetic basis of familial forms of idiopathic pulmonary fibrosis is not understood.

Telomeres are DNA-protein structures that protect chromosome ends. Telomeres shorten successively with each cell division and short telomeres ultimately activate a DNA damage response that leads to cell death or permanent cell cycle arrest (48-50). Accordingly, this biology has implicated telomere shortening in degenerative age-related disease. Telomerase is a specialized polymerase that adds telomere repeats to the ends of chromosomes. It has two essential components: a catalytic component, telomerase reverse transcriptase (hTERT), and an RNA component (hTR); the latter provides the template for nucleotide addition by hTERT. The addition of telomeric repeats (a repeat comprising the six nucleotides—TTAGGG complementary to the template in hTR) to the ends of the chromosome partly offsets the shortening that occurs during DNA replication. Telomeres shorten with each cell division and ultimately activate a DNA damage response that leads to apoptosis or cell-cycle arrest. Telomere length thus limits the replicative capacity of tissues and has been implicated in age-related disease.

Dyskeratosis congenita is a rare hereditary disorder initially described on the basis of a triad of mucocutaneous manifestations: skin hyperpigmentation, oral leukoplakia, and nail dystrophy. The most common cause of death in patients with dyskeratosis congenita is bone marrow failure due to aplastic anemia. Pulmonary disease is present in 20% of patients and is the second most common cause of death. The X-linked form of dyskeratosis congenita is severe, and is associated with mutations in the DKC1 gene. Autosomal dominant cases of dyskeratosis congenita are rare, and can present later in adulthood, and often lack the classic skin manifestations. In some families, the hematopoietic defects develop first, implying that despite the originally given name, the dyskeratosis is not canonical. Heterozygous mutations in hTR and hTERT, the essential components of telomerase, underlie the genetic defect in families with dominant inheritance, indicating that half the usual dose of telomerase is inadequate for telomere maintenance, and tissues of high turnover, such as the bone marrow, are susceptible. In autosomal dominant dyskeratosis congenita, anticipation can be seen in which phenotypes present earlier and more severely in successive generations. Moreover, since the consequences of carrying mutant telomerase genes can appear in adulthood as either idiopathic pulmonary fibrosis or aplastic anemia without dyskeratosis, the consideration of such cases as part of a syndrome of telomere shortening may heighten the index of suspicion and facilitate diagnosis.

The development of genetic detection, such as screening, and diagnostic methods that would allow for early diagnosis of telomere associated diseases or disorders, would facilitate timely therapeutic intervention. In certain cases, timely intervention would allow therapy before damage to organs and tissues, or even death, occurs. Furthermore, the identification of genetic alterations associated with telomere associated diseases or disorders, and the understanding of the role that they play in the development of the pathology are important for new and improved therapeutic strategies.

### SUMMARY OF THE INVENTION

As described below, the present invention features methods of detecting, diagnosing a presence or a predisposition to, or determining the risk for a subject to develop telomere-associated diseases or disorders. The methods include detecting the presence or absence of an alteration in a nucleic acid in a sample. The methods also include determining telomere length. In certain cases, average telomere length is a surrogate marker for a telomere-associated disease or disorder.

In a first aspect, the invention provides a method of diagnosing a telomere-associated disease or disorder in a subject, comprising providing a sample from the subject; and analyzing all or part of a nucleic acid sequence corresponding to one or more of the telomere genes in the sample for the presence of one or more alterations, wherein the presence of at least one alteration is indicative of an increased risk of the presence of a telomere-associated disease or disorder.

In one embodiment, the telomere associated gene is selected from hTERT (telomerase reverse transcriptase) and hTR (telomerase RNA). In another embodiment, the method further comprises the step of determining the average telomere length.

In another aspect, the invention features a method of diagnosing a telomere-associated disease or disorder in a subject, comprising providing a sample from a the subject; and determining the average telomere length in the sample compared to a control, wherein a shorter average telomere length is indicative of an increased risk of the presence of a telomere-associated disease or disorder.

In an embodiment of any one of the above aspects, the telomere-associated disease or disorder is selected from the group consisting of dyskeratosis congenita, hematopoietic defects, idiopathic pulmonary fibrosis, and cryptogenic liver cirrhosis.

In another aspect of the above method, the telomere length is determined by a method selected from the group comprising: terminal restriction fragment analysis, fluorescent in situ hybridization, flow cytometry, and quantitative polymerase chain reaction (PCR). In a further aspect of the above method, the average telomere length is determined in leukocytes. In another related embodiment, the average telomere length is a surrogate marker for a telomere-associated disease or disorder. In still another embodiment, the average telomere length is predictive of a telomerase alteration.

In one embodiment, the one or more alterations is selected from the group consisting of: SEQ ID NO: 1, SEQ ID NO: 2, SEQ ID NO: 3, SEQ ID NO: 4, SEQ ID NO: 5, and SEQ ID NO: 6.

In another embodiment, the nucleic acid sequence is selected from the group consisting of: genomic DNA, cDNA, mRNA, and chromosomes.

In a related embodiment, the analysis further comprises an amplification step.

In still a further embodiment, the analysis further comprises a hybridization step using at least one primer set specific for the sequence to identify sequence alterations. In a related embodiment, the primer set comprises a forward primer selected from the group consisting of SEQ ID NOs: 11-27 and a reverse primer selected from the group consisting of SEQ ID NOs: 28-42. In still a further embodiment, the primer set comprises a reverse primer selected from the group consisting of SEQ ID NOs: 28-42.

In one embodiment, the presence of an alteration is detected by comparison with the corresponding non-mutated wildtype sequence.

In a further embodiment, the analysis is carried out by sequencing.

In another further embodiment, all or part of the nucleic acid sequence of the telomere-associated genes is amplified prior to detection of said at least one alteration.

In a related embodiment, the amplification is carried out by PCR or PCR-like amplification.

In another aspect, the invention features a method of diagnosing a presence or a predisposition to a telomere-associated disease or disorder in a subject, comprising providing a sample from the subject; and analyzing all or part of a nucleic acid sequence corresponding to one or more telomere genes in the sample for the presence of one or more alterations in the telomere genes, wherein the analyzing comprises amplifying all or part of the nucleic acid sequence of the telomere genes prior to detection, said amplifying being performed with at least one primer consisting of the sequence of any one of SEQ ID NOs: 11-42, wherein the presence of at least one alteration is indicative of an increased risk for the presence or predisposition to a telomere-associated disease or disorder.

In still another aspect, the invention features a method of detecting the presence or absence of an alteration in a nucleic acid in a sample, comprising analyzing all or part of a nucleic acid corresponding to a telomere associated gene in the sample and a control sample to determine whether one or more alterations are present in the sample nucleic acid; and determining the presence or absence of one or more alterations in the sample nucleic acid compared to the control sample nucleic acid.

In one embodiment, the telomere associated gene is selected hTERT and hTR.

In another embodiment, the presence of one or more alterations in the telomere associated gene is indicative a disease or disorder selected from: dyskeratosis congenita, hematopoietic defects, idiopathic pulmonary fibrosis, idiopathic interstitial pneumonia, and cryptogenic liver cirrhosis.

In another aspect, the invention features a method of diagnosing of diagnosing a presence or a predisposition to a telomere-associated disease or disorder in a subject, comprising providing a sample from a the subject; and determining the average length of telomeres in the sample compared to a control, wherein a shorter average telomere length is indicative of an increased risk for the presence or predisposition to a telomere-associated disease or disorder.

In still another aspect, the invention features a method for determining the risk for a subject to develop a telomere-associated disease or disorder, comprising detecting the presence or absence of an alteration in a telomere gene in a test sample obtained from the subject, comprising analyzing all or a part of a nucleic acid corresponding to a telomere gene in the sample and a control sample to determine whether at least one alteration is present in the test sample nucleic acid wherein the control sample comprises a telomere gene; and determining the presence or absence of one or more alterations in the telomere gene in the test sample compared to the telomere gene in the control sample nucleic acids, wherein the presence of one or more alterations in the telomere gene in the test sample compared to the control correlates with increased risk to develop a telomere associated disease or disorder.

In another aspect, the invention features a method for determining the risk for a subject to develop a telomere-associated disease or disorder, comprising determining the average length of telomeres in a test sample obtained from the subject, comprising providing a sample from a the subject; and determining the average length of telomeres in the sample compared to a control, wherein a shorter average telomere length is indicative of an increased risk to develop a telomere associated disease or disorder.

In one aspect of any one of the above methods, the telomere-associated disease or disorder is selected from the group consisting of: dyskeratosis congenita, hematopoietic defects, idiopathic pulmonary fibrosis, and cryptogenic liver cirrhosis.

In another aspect, the above methods further comprise the step of determining the average telomere length.

In another aspect of the above methods, telomere length is determined by a method selected from the group comprising: terminal restriction fragment analysis, fluorescent in situ hybridization, flow cytometry, and quantitative PCR.

In one embodiment, telomere length is determined in leukocytes.

In another embodiment, the average telomere length is a surrogate marker for a telomere-associated disease or disorder. In a related embodiment, a telomere length of less than 5 kilobases is predictive of a telomerase alteration.

In another embodiment, the one or more alterations is selected from the group consisting of: SEQ ID NO: 3, SEQ ID NO: 4, SEQ ID NO: 5, SEQ ID NO: 6, SEQ ID NO: 7, and SEQ ID NO: 8.

In still another related embodiment, the nucleic acid sequence is selected from the group consisting of: genomic DNA, cDNA, mRNA, and chromosomes.

In another embodiment, the analysis further comprises amplification and hybridization using at least one primer set specific for the sequence to identify sequence alterations.

In a further embodiment, the primer set comprises a forward primer selected from the group consisting of SEQ ID NOs: 11-27 and a reverse primer selected from the group consisting of SEQ ID NOs: 28-48.

In a related embodiment, the presence of an alteration is detected by comparison with the corresponding nonmutated natural sequence.

In another embodiment, the analysis is carried out by sequencing.

In one embodiment of the above aspects, all or part of the nucleic acid sequence of the telomere-associated genes is amplified prior to detection of said at least one alteration.

In a further embodiment, the amplification is carried out by PCR or PCR-like amplification.

In one embodiment of the above aspects, the nucleic acid alteration in the telomerase gene is detected by screening for a deletion mutation.

In another embodiment of the above aspects, the nucleic acid alteration in the telomerase gene is detected by screening for a point mutation.

In one embodiment of the above aspects, the nucleic acid alteration in the telomerase gene is detected by screening for an insertion.

In another embodiment of the above aspects, the nucleic acid alteration in the telomerase gene is detected by screening for an inversion.

In one embodiment of the above aspects, the nucleic acid alteration in the telomerase gene is detected by screening for a missense mutation.

In another embodiment of the above aspects, the nucleic acid alteration in the telomerase gene is detected by fluorescence in situ hybridization of a telomere gene or part thereof with nucleic acid probes which comprise the telomere gene.

In one embodiment of the above aspects, the alteration is a change in copy number. In a further embodiment, the alteration is detected by fluorescence in situ hybridization.

In a further embodiment of the above aspects, the alteration in the nucleic acid occurs in the telomerase essential N-terminal domain of SEQ ID NO: 7 (hTERT).

In another further embodiment of the above aspects, the alteration in the nucleic acid occurs in the C-terminal domain of SEQ ID NO: 7.

In a further embodiment of the above aspects, the alteration in the nucleic acid occurs in the pseudoknot domain of SEQ ID NO: 8 (hTR]

In another further embodiment of the above aspects, the alteration in the nucleic acid results in a polypeptide comprising an amino acid sequence that differs from SEQ ID NO: 7 (hTERT) by one or more alterations selected from the group consisting of: Leu55Gln, Thr111Met, codon 112delC, intervening sequence between exons IVS1+1G to A, and IVS9-2A to C.

In one embodiment of the above aspects, the alteration in the nucleic acid results in a polypeptide comprising an amino acid sequence that differs from SEQ ID NO: 8 (hTR) by an alteration at residue 98 comprising a G to A substitution.

In yet another embodiment of the above aspects, the method further comprises a step of determining telomere activity.

In one embodiment of the above aspects, the method is used to determine a course of screening or treatment.

In yet another embodiment of the above aspects, the method further comprises administration of a therapeutic agent. In another embodiment, the therapeutic agent can be a chemotherapeutic, an apoptotic agent, and a cell proliferation inhibitor.

In one embodiment of the above aspects, the subject is a mammal

In another embodiment, the mammal is a human.

In another aspect, the invention features an oligonucleotide primer for detecting in a subject a genetic risk for developing a telomere-associated disease or disorder comprising the sequence selected from the group consisting of: SEQ ID NOs: 11-42.

In still another aspect, the invention features a primer set for detecting in a subject a genetic risk for developing a telomere-associated disease or disorder, the primer set having a forward primer selected from the group consisting of: SEQ ID NOs: 11-27 and a reverse primer selected from the group consisting of: SEQ ID NOs: 28-42.

In another aspect, the invention features a kit for use in diagnosing a telomere-associated disease or disorder in a subject or diagnosing the presence or a predisposition a telomere-associated disease or disorder in a subject, and instructions for use.

In one embodiment, the kit further comprises the primer set the aspect of the invention, and instructions for use.

Other features and advantages of the invention will be apparent from the detailed description, and from the claims.

### BRIEF DESCRIPTION OF THE DRAWINGS

FIG. 1 shows chromatograms of telomerase mutations in probands from Families A-F.

FIG. 2 shows pedigrees of six probands with telomerase mutations.

FIG. 3 (A and B) is two graphs showing telomere length in mutation carriers and their relatives. Panel A shows the average length of telomeres in lymphocytes in eight carriers and seven non-carriers of the genetic mutation, and Panel B shows telomere length as a function of age.

FIG. 4 (A-E) shows the biochemical consequences of telomerase mutations in probands. Panel A shows conserved domains of hTERT with missense mutations, as indicated. Panel B shows the secondary structure of hTR, with the site of the mutation indicated by an asterisk. Panel C shows the telomerase activity of mutant hTERT and hTR alleles, as measured by the direct assay and the intensity and pattern of the repetitive ladder. Panel D shows the quantitation of telomerase activity at the second major band, as indicated by the arrowhead in Panel C. Panel E shows the results of an RT-PCR assay across exons 9 through 11 from a subject with an hTERT 9-2 A-4C mutation.

FIG. 5 (A-H) shows high-resolution computed tomographic images of the midlung (Panel A-Panel D) and bases (Panel E-Panel H) in probands in four Families.

FIG. 6 (A-D) is four graphs that show telomere length in lymphocytes from IIP patients and families with known telomerase mutations compared to controls. In Panel A, the graph shows that IIP patients have shorter telomeres than age-matched controls (p<0.0001, Wilcoxon signed rank). 60 of 62 (97%) of IIP patients have telomeres shorter than the median of healthy controls (p<0.0001). Of the 62 IIP patients, 50 (81%) carried the diagnosis of IPF. Panel B is a detailed view of A: * refers to a 77 year old IPF patient with hTR 325G→T mutation, § refers to a patient with very short telomeres who had chronic unexplained thrombocytopenia with macrocytosis both suggestive of subclinical aplastic anemia, ‡ denotes two individuals with both IPF and cryptogenic liver cirrhosis who have short telomeres. Ten percent of IIP patients (6 of 62) have short telomeres below the first percentile; a range predictive of the presence of a telomerase mutation. Panel C shows the telomere length from 45 individuals from 10 families with known mutations in hTERT, hTR and DKC1. Mutation carriers harbored hTERT Leu55Gln (n=2), hTERT IVS1+1G→A (n=1), hTERT IVS9-2A→C (n=1), hTERT Val791Ile/Val867Met (n=7), hTERT Lys902Asn (n=4), hTERT Thr1110Met (n=2), hTR98A (n=1), hTR204G (n=2), DKC1 Gln31Glu (n=4). Panel D is a bar graph illustrating the mean difference in telomere length from the median of age-matched healthy controls (delta Telomere length). Compared with non-carriers whose telomere length was similar to controls (p=0.304, Wilcoxon signed rank), both sporadic IIP patients and known telomerase mutation carriers had shorter telomeres (p<0.0001 for both).

FIG. 7 (A-C) is three panels that show germline mutation in hTR in an IPF patient with no family history. The results in Panel A show hTR 325 G→T lies in the P5 helix of hTR and is expected to compromise function by disrupting helical integrity. The results in Panel B show telomerase activity of mutant hTR shows compromised catalysis as shown by the intensity of the repeat ladder compared with wildtype with quantitation shown in C.

FIG. 8 (A-D) shows telomere length in the alveolar epithelium using quantitative in situ FISH. The result shows that lung cells from patients with sporadic pulmonary fibrosis with a confirmed biopsy showing usual interstitial pneumonia have short telomeres. Panel A shows representative images of nuclei of surfactant positive C cells (cytoplasmic staining in green) from an individual with no known lung disease showing bright telomere signals after hybridizing with PNA labeled telomere probe (pink). In contrast, alveolar cells from a patient with IPF/UIP have significantly shorter telomeres as seen by the dim or absent telomere signal in Panel B. Panel C shows telomere signals from individuals with normal lungs, sporadic IPF, and known hTERT (n=6) or hTR mutation (n=1) carriers (42) with age at the time of biopsy are shown below. In this small dataset, there is a trend toward decreasing telomere length with age. The results in Panel D show IPF patients (with or without a family history) have shorter telomeres in alveolar cells than controls (p-values using two-sided Student t-test).

FIG. 9 (A-F) shows representative imaging and pathology from patients diagnosed with both idiopathic pulmonary fibrosis and cryptogenic liver cirrhosis. Computed tomography panels A and D show honeycomb changes of IPF in the lung bases. B and E show representative abnormalities in the same patients with evidence of decompensated cirrhosis with hypersplenism and portal hypertension in B and nodular and abnormal liver contour in E associated with operative description of cirrhotic liver. C and F. Reticulin stains of liver explants from patient shown in A and B and of a second Johns Hopkins patient who underwent liver transplant 3 years prior to the diagnosis of IPF. The fibrosis on the background of cirrhotic lobules is prominent in the interstitial and perivascular space.

FIG. 10 shows telomerase activity of non-synonymous hTERT variants identified in both sporadic IPF patients and in healthy controls. Telomerase activity of hTERT Ala279Thr, His412Tyr, Ala1062Thr was intact showing no compromise in catalysis or processivity as quantitated by the direct assay both in vitro and after reconstitution in VA13 cells (not shown). In contrast hTERT Leu55Gln previously identified in a family with inherited IPF shows decreased activity as described (42).

### DETAILED DESCRIPTION OF THE INVENTION

Telomeres are DNA-protein structures that protect chromosome ends. Because of the end-replication problem, telomeres shorten with each cell division and ultimately signal a DNA damage response that leads to cell death or permanent cell cycle arrest. Short telomeres have thus been implicated in age-related disease. Telomerase is a specialized reverse transcriptase responsible for telomere addition that offsets the natural shortening that occurs with each cell division. Telomerase has two essential components: hTERT, telomerase reverse transcriptase and hTR, telomerase RNA. It is a finding of the instant invention that germline mutations in these telomerase components are associated with a complex phenotype of dyskeratosis congenita in humans. These phenotypes include but are not exclusive to: hematopoietic defects, idiopathic pulmonary fibrosis or idiopathic interstitial pneumonia, cryptogenic liver cirrhosis/fibrosis, an increased cancer predisposition, childhood osteoporosis, and premature hair graying. These phenotypes can appear isolated in patients and families who have mutations in telomerase. It is also conceivable that mutations in other, yet unidentified, components of the telomere pathway or of the telomerase enzyme may account for the phenotypes of dyskeratosis congenita. Diagnostic testing in families with these clinical features may allow for tailored therapies as well as for risk assessment in asymptomatic family members. These data, combined with telomere length measurements by flow-cytometry based fluorescence in situ hybridization provide a powerful tool to assess disease risk.

The present invention features methods of detecting, diagnosing a presence or a predisposition to, or determining the risk for a subject to develop telomere-associated diseases or disorders. The methods include detecting the presence or absence of an alteration in a nucleic acid in a sample. The methods also include determining telomere length. In certain cases, average telomere length is a surrogate marker for a telomere-associated disease or disorder.

### Definitions

The following definitions are provided for specific terms which are used in the following written description.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used in the specification and claims, the singular form “a”, “an” and “the” includes plural references unless the context clearly dictates otherwise.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “agent” or “therapeutic agent” as used herein is meant to refer to a polypeptide, polynucleotide, or fragment, or analog thereof, small molecule, or other biologically active molecule that has therapeutic effect in a subject.

The term “alteration” as used herein is meant to refer to a change in a gene. As used herein, an alteration includes a mutation. In certain examples, an alteration can be a deletion mutation, a point mutation, an insertion, an inversion, a missense mutation, or a change in copy number; however any alteration is possible according to the invention as described herein.

The phrase “average telomere length” is meant to refer to a measure of telomere length in a population of cells. In certain embodiments, average telomere length of a test sample, e.g. a subject suspected of having a telomere associated disease or disorder is compared to a control subject.

The term “control” is meant to refer to a standard or reference condition.

The term “gene” is meant to refer to a segment of nucleic acid that contains the information necessary to produce a functional RNA product. A gene usually contains regulatory regions dictating under what conditions the RNA product is made, transcribed regions dictating the sequence of the RNA product, and/or other functional sequence regions. A gene may be transcribed to produce an mRNA molecule, which contains the information necessary for translation into the amino acid sequence of the resulting protein.

The phrase “telomere associate disease or disorder” is meant to refer to any disease, condition, or disorder that is caused by an alteration in a gene associated with the telomere or the telomere pathway. A telomere associated disease or disorder can also refer to any disease, condition or disorder that is caused by shortening of the telomere. In certain preferred examples, a telomere associated disease or disorder can be dyskeratosis congenita, hematopoietic defects, idiopathic pulmonary fibrosis, idiopathic interstitial pneumonia, or cryptogenic liver cirrhosis.

The term “telomere associated gene” is meant to refer to a gene that is part of the telomere complex or a gene that encodes any member of the telomere pathway. Exemplary telomere associated genes include, but are not limited to hTERT (telomerase reverse transcriptase) and hTR (telomerase RNA).

The term “nucleic acid” is meant to refer to both RNA and DNA, including cDNA, genomic DNA, mRNA, synthetic DNA and chimeras of RNA and DNA. The nucleic acid can be double-stranded or single-stranded. Where single-stranded, the nucleic acid can be a sense strand or an antisense strand. The nucleic acid can be synthesized using nucleotide analogs or derivatives (e.g., inosine or phosphorothioate nucleotides).

The term “dyskeratosis congenita” is meant to refer to a hereditary disorder with features that include, but are not limited to, cutaneous pigmentation, dystrophy of the nails, leukoplakia of the oral mucosa and low blood counts. The term “idiopathic pulmonary fibrosis” is meant to refer to a progressive disease characterized by irreversible fibrosis.

The term “idiopathic pulmonary fibrosis” is meant to refer to a progressive disease characterized by irreversible fibrosis that has a clinically recognizable radiographic and pathologic appearance. This is outlined in the American Thoracic Society in 2002 (American Thoracic Society/European Respiratory Society International Multidisciplinary Consensus Classification of the Idiopathic Interstitial Pneumonias. This joint statement of the American Thoracic Society (ATS), and the European Respiratory Society (ERS) was adopted by the ATS board of directors, June 2001 and by the ERS Executive Committee, June 2001. Am J Respir Crit Care Med 2002; 165(2):277-304.).

The term “idiopathic interstitial pneumonia” is meant refers to interstitial lung diseases that have no known etiology. The term can, in certain embodiments, be as defined by American Thoracic Society/European Respiratory Society International Multidisciplinary Consensus Classification of the Idiopathic Interstitial Pneumonias. This joint statement of the American Thoracic Society (ATS), and the European Respiratory Society (ERS) was adopted by the ATS board of directors, June 2001 and by the ERS Executive Committee, June 2001. Am J Respir Crit Care Med 2002; 165(2):277-304. Idiopathic Pulmonary Fibrosis is the most common of all idiopathic interstitial pneumonias comprising more than 70% of cases.

The term “cryptogenic liver cirrhosis” is meant to refer to a condition whereby the etiology of liver failure (both compensated and uncompensated) is not explained by autoimmune, infectious, infiltrative or other processes.

The term “part” or “part of a nucleic acid sequence” is meant to refer to a fragment of a nucleic acid molecule. In certain examples, the fragment can be of a polypeptide. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

The term “telomerase reverse transcriptase” or hTERT is meant to refer to a ribonucleoprotein polymerase that maintains telomere ends by addition of the telomere repeat TTAGGG. TERT consists of a protein component with reverse transcriptase activity, and an RNA component which serves as a template for the telomere repeat. In certain embodiments, the sequence of full-length native hTERT is represented by GenBank Accession No. AF015950.

The term “telomerase RNA” or hTR is meant to refer to the telomerase RNA component. In certain cases, hTR is known as hTERC. In certain embodiments, the sequence of full-length native hTR is represented by GenBank Accession No. U85256/NR_{—}001566.

The term “subject” is intended to include vertebrates, preferably a mammal. Subjects according to the present invention can include humans as well as animals. Suitable subjects include without limitation non-human primates, dogs, cats, horses, cattle, pigs, sheep, goats, guinea pigs, mice, rats, rabbits and chickens. The subject can be homozygous or heterozygous for the mutations described herein. Mammals include, but are not limited to, humans.

### Methods of the Invention

As described herein the invention generally features methods of detection, methods of diagnosing a presence or a predisposition to, or determining the risk for a subject to develop telomere-associated diseases or disorders. The methods include detecting the presence or absence of an alteration in a nucleic acid in a sample. The methods also include determining telomere length. In certain cases, average telomere length is a surrogate marker for a telomere-associated disease or disorder.

### Telomeres and Telomerase

Normal human somatic cells, unlike cancer cells, undergo only a limited number of cell divisions and eventually stop dividing through the process termed cellular senescence or replicative senescence (see, e.g. Goldstein, 249 Science 1129, 1990; Hayflick and Moorehead, 25 Exp. Cell Res. 585, 1961; Ohno, 11 Mech. Aging Dev. 179, 1979; Ham and McKeehan, (1979) “Media and Growth Requirements”, W. B. Jacoby and I. M. Pastan (eds), in: Methods in Enzymology, Academic Press, N.Y., 58:44-93, all of which are incorporated by reference in their entireties herein. Cellular senescence can be a tumor suppressive mechanism in human cells, and immortalization is a rate-limiting step for cancer cells to develop and grow.

It has long been recognized that complete replication of the ends of eukaryotic chromosomes requires specialized cell components (Watson, 1972, Nature New Biol., 239:197; Olovnikov, 1973, J. Theor. Biol., 41:181). Replication of a linear DNA strand by conventional DNA polymerase requires an RNA primer, and can proceed only 5′ to 3′. When the RNA bound at the extreme 5′ ends of eukaryotic chromosomal DNA strands is removed, a gap is introduced, leading to a progressive shortening of daughter strands with each round of replication. This shortening of telomeres, the protein-DNA structures physically located on the ends of chromosomes, is thought to account for the phenomenon of cellular senescence or aging of normal human somatic cells in vitro and in vivo. The maintenance of telomeres is a function of a telomere-specific DNA polymerase known as telomerase. Telomerase is a ribonucleoprotein (RNP) that uses a portion of its RNA moiety as a template for telomeric DNA synthesis (Morin, 1997, Eur. J. Cancer 33:750). The length and integrity of telomeres and the telomerase expression status of a cell is thus related to entry of a cell into a senescent stage (i.e., loss of proliferative capacity), or the ability of a cell to escape senescence, i.e., to become immortal.

Consistent with the relationship of telomeres and telomerase to the proliferative capacity of a cell (i.e., the ability of the cell to divide indefinitely), telomerase activity is detected in immortal cell lines and an extraordinarily diverse set of tumor tissues, but is not detected (i.e., was absent or below the assay threshold) in normal somatic cell cultures or normal tissues adjacent to a tumor (see, U.S. Pat. Nos. 5,629,154; 5,489,508; 5,648,215; and 5,639,613; see also, Morin, 1989, Cell 59: 521; Shay and Bacchetti 1997, Eur. J. Cancer 33:787; Kim et al., 1994, Science 266:2011; Counter et al., 1992, EMBO J. 11:1921; Counter et al., 1994, Proc. Natl. Acad. Sci. U.S.A. 91, 2900; Counter et al., 1994, J. Virol. 68:3410). Moreover, a correlation between the level of telomerase activity in a tumor and the likely clinical outcome of the patient has been reported (e.g., U.S. Pat. No. 5,639,613, supra; Langford et al., 1997, Hum. Pathol. 28:416).

Telomerase is a ribonucleoprotein complex (RNP) comprising an RNA component and a catalytic protein component. Among multiple protein components and an RNA component of human telomerase, the catalytic protein subunit, human telomerase reverse transcriptase (hTERT), is the key determinant of the enzymatic activity of human telomerase. Telomerase maintains telomere ends by addition of the telomere repeat TTAGGG. Telomerase expression plays a role in cellular senescence, as it is normally repressed in postnatal somatic cells resulting in progressive shortening of telomeres. Deregulation of telomerase expression in somatic cells may be involved in oncogenesis. Studies in mouse suggest that telomerase also participates in chromosomal repair, since de novo synthesis of telomere repeats may occur at double-stranded breaks. Alternatively spliced variants encoding different isoforms of telomerase reverse transcriptase have been identified; the full-length sequence of some variants has not been determined. Alternative splicing at this locus is thought to be one mechanism of regulation of telomerase activity. hTERT has been cloned, and protein, cDNA and genomic sequences determined. See, e.g., Nakamura et al., 1997, Science 277:955, and U.S. Pat. Nos. 6,475,789 and 6,166,178. The sequence of a full-length wild type hTERT has been deposited in GenBank (Accession No. AF015950). The sequence of Homo sapiens telomerase reverse transcriptase (hTERT) is shown below and comprises SEQ ID NO: 7 (nucleotide) and SEQ ID NO: 9 (amino acid).

| SEQ ID NO: 7 | |
|---|---|
| 1 | gcagcgctgc gtcctgctgc gcacgtggga agccctggcc ccggccaccc ccgcgatgcc |
| 61 | gcgcgctccc cgctgccgag ccgtgcgctc cctgctgcgc agccactacc gcgaggtgct |
| 121 | gccgctggcc acgttcgtgc ggcgcctggg gccccagggc tggcggctgg tgcagcgcgg |
| 181 | ggacccggcg gctttccgcg cgctggtggc ccagtgcctg gtgtgcgtgc cctgggacgc |
| 241 | acggccgccc cccgccgccc cctccttccg ccaggtgtcc tgcctgaagg agctggtggc |
| 301 | ccgagtgctg cagaggctgt gcgagcgcgg cgcgaagaac gtgctggcct tcggcttcgc |
| 361 | gctgctggac ggggcccgcg ggggcccccc cgaggccttc accaccagcg tgcgcagcta |
| 421 | cctgcccaac acggtgaccg acgcactgcg ggggagcggg gcgtgggggc tgctgctgcg |
| 481 | ccgcgtgggc gacgacgtgc tggttcacct gctggcacgc tgcgcgctct ttgtgctggt |
| 541 | ggctcccagc tgcgcctacc aggtgtgcgg gccgccgctg taccagctcg gcgctgccac |
| 601 | tcaggcccgg cccccgccac acgctagtgg accccgaagg cgtctgggat gcgaacgggc |
| 661 | ctggaaccat agcgtcaggg aggccggggt ccccctgggc ctgccagccc cgggtgcgag |
| 721 | gaggcgcggg ggcagtgcca gccgaagtct gccgttgccc aagaggccca ggcgtggcgc |
| 781 | tgcccctgag ccggagcgga cgcccgttgg gcaggggtcc tgggcccacc cgggcaggac |
| 841 | gcgtggaccg agtgaccgtg gtttctgtgt ggtgtcacct gccagacccg ccgaagaagc |
| 901 | cacctctttg gagggtgcgc tctctggcac gcgccactcc cacccatccg tgggccgcca |
| 961 | gcaccacgcg ggccccccat ccacatcgcg gccaccacgt ccctgggaca cgccttgtcc |
| 1021 | cccggtgtac gccgagacca agcacttcct ctactcctca ggcgacaagg agcagctgcg |
| 1081 | gccctccttc ctactcagct ctctgaggcc cagcctgact ggcgctcgga ggctcgtgga |
| 1141 | gaccatcttt ctgggttcca ggccctggat gccagggact ccccgcaggt tgccccgcct |
| 1201 | gccccagcgc tactggcaaa tgcggcccct gtttctggag ctgcttggga accacgcgca |
| 1261 | gtgcccctac ggggtgctcc tcaagacgca ctgcccgctg cgagctgcgg tcaccccagc |
| 1321 | agccggtgtc tgtgcccggg agaagcccca gggctctgtg gcggcccccg aggaggagga |
| 1381 | cacagacccc cgtcgcctgg tgcagctgct ccgccagcac agcagcccct ggcaggtgta |
| 1441 | cggcttcgtg cgggcctgcc tgcgccggct ggtgccccca ggcctctggg gctccaggca |
| 1501 | caacgaacgc cgcttcctca ggaacaccaa gaagttcatc tccctgggga agcatgccaa |
| 1561 | gctctcgctg caggagctga cgtggaagat gagcgtgcgg gactgcgctt ggctgcgcag |
| 1621 | gagcccaggg gttggctgtg ttccggccgc agagcaccgt ctgcgtgagg agatcctggc |
| 1681 | caagttcctg cactggctga tgagtgtgta cgtcgtcgag ctgctcaggt ctttctttta |
| 1741 | tgtcacggag accacgtttc aaaagaacag gctctttttc taccggaaga gtgtctggag |
| 1801 | caagttgcaa agcattggaa tcagacagca cttgaagagg gtgcagctgc gggagctgtc |
| 1861 | ggaagcagag gtcaggcagc atcgggaagc caggcccgcc ctgctgacgt ccagactccg |
| 1921 | cttcatcccc aagcctgacg ggctgcggcc gattgtgaac atggactacg tcgtgggagc |
| 1981 | cagaacgttc cgcagagaaa agagggccga gcgtctcacc tcgagggtga aggcactgtt |
| 2041 | cagcgtgctc aactacgagc gggcgcggcg ccccggcctc ctgggcgcct ctgtgctggg |
| 2101 | cctggacgat atccacaggg cctggcgcac cttcgtgctg cgtgtgcggg cccaggaccc |
| 2161 | gccgcctgag ctgtactttg tcaaggtgga tgtgacgggc gcgtacgaca ccatccccca |
| 2221 | ggacaggctc acggaggtca tcgccagcat catcaaaccc cagaacacgt actgcgtgcg |
| 2281 | tcggtatgcc gtggtccaga aggccgccca tgggcacgtc cgcaaggcct tcaagagcca |
| 2341 | cgtctctacc ttgacagacc tccagccgta catgcgacag ttcgtggctc acctgcagga |
| 2401 | gaccagcccg ctgagggatg ccgtcgtcat cgagcagagc tcctccctga atgaggccag |
| 2461 | cagtggcctc ttcgacgtct tcctacgctt catgtgccac cacgccgtgc gcatcagggg |
| 2521 | caagtcctac gtccagtgcc aggggatccc gcagggctcc atcctctcca cgctgctctg |
| 2581 | cagcctgtgc tacggcgaca tggagaacaa gctgtttgcg gggattcggc gggacgggct |
| 2641 | gctcctgcgt ttggtggatg atttcttgtt ggtgacacct cacctcaccc acgcgaaaac |
| 2701 | cttcctcagg accctggtcc gaggtgtccc tgagtatggc tgcgtggtga acttgcggaa |
| 2761 | gacagtggtg aacttccctg tagaagacga ggccctgggt ggcacggctt ttgttcagat |
| 2821 | gccggcccac ggcctattcc cctggtgcgg cctgctgctg gatacccgga ccctggaggt |
| 2881 | gcagagcgac tactccagct atgcccggac ctccatcaga gccagtctca ccttcaaccg |
| 2941 | cggcttcaag gctgggagga acatgcgtcg caaactcttt ggggtcttgc ggctgaagtg |
| 3001 | tcacagcctg tttctggatt tgcaggtgaa cagcctccag acggtgtgca ccaacatcta |
| 3061 | caagatcctc ctgctgcagg cgtacaggtt tcacgcatgt gtgctgcagc tcccatttca |
| 3121 | tcagcaagtt tggaagaacc ccacattttt cctgcgcgtc atctctgaca cggcctccct |
| 3181 | ctgctactcc atcctgaaag ccaagaacgc agggatgtcg ctgggggcca agggcgccgc |
| 3241 | cggccctctg ccctccgagg ccgtgcagtg gctgtgccac caagcattcc tgctcaagct |
| 3301 | gactcgacac cgtgtcacct acgtgccact cctggggtca ctcaggacag cccagacgca |
| 3361 | gctgagtcgg aagctcccgg ggacgacgct gactgccctg gaggccgcag ccaacccggc |
| 3421 | actgccctca gacttcaaga ccatcctgga ctgatggcca cccgcccaca gccaggccga |
| 3481 | gagcagacac cagcagccct gtcacgccgg gctctacgtc ccagggaggg aggggcggcc |
| 3541 | cacacccagg cccgcaccgc tgggagtctg aggcctgagt gagtgtttgg ccgaggcctg |
| 3601 | catgtccggc tgaaggctga gtgtccggct gaggcctgag cgagtgtcca gccaagggct |
| 3661 | gagtgtccag cacacctgcc gtcttcactt ccccacaggc tggcgctcgg ctccacccca |
| 3721 | gggccagctt ttcctcacca ggagcccggc ttccactccc cacataggaa tagtccatcc |
| 3781 | ccagattcgc cattgttcac ccctcgccct gccctccttt gccttccacc cccaccatcc |
| 3841 | aggtggagac cctgagaagg accctgggag ctctgggaat ttggagtgac caaaggtgtg |
| 3901 | ccctgtacac aggcgaggac cctgcacctg gatgggggtc cctgtgggtc aaattggggg |
| 3961 | gaggtgctgt gggagtaaaa tactgaatat atgagttttt cagttttgaa aaaaa |

SEQ ID NO: 9
MPRAPRCRAVRSLLRSHYREVLPLATFVRRLGPQGWRLVQRGDPAAFRA
LVAQCLVCVPWDARPPPAAPSFRQVSCLKELVARVLQRLCERGAKNVLA
FGFALLDGARGGPPEAFTTSVRSYLPNTVTDALRGSGAWGLLLRRVGDD
VLVHLLARCALFVLVAPSCAYQVCGPPLYQLGAATQARPPPHASGPRRR
LGCERAWNHSVREAGVPLGLPAPGARRRGGSASRSLPLPKRPRRGAAPEP
ERTPVGQGSWAHPGRTRGPSDRGFCVVSPARPAEEATSLEGALSGTRHSH
PSVGRQHHAGPPSTSRPPRPWDTPCPPVYAETKHFLYSSGDKEQLRPSFLL
SSLRPSLTGARRLVETIFLGSRPWMPGTPRRLPRLPQRYWQMRPLFLELLG
NHAQCPYGVLLKTHCPLRAAVTPAAGVCAREKPQGSVAAPEEEDTDPRR
LVQLLRQHSSPWQVYGFVRACLRRLVPPGLWGSRHNERRFLRNTKKFISL
GKHAKLSLQELTWKMSVRDCAWLRRSPGVGCVPAAEHRLREEILAKFLH
WLMSVYVVELLRSFFYVTETTFQKNRLFFYRKSVWSKLQSIGIRQHLKRV
QLRELSEAEVRQHREARPALLTSRLRFIPKPDGLRPIVNMDYVVGARTFR
REKRAERLTSRVKALFSVLNYERARRPGLLGASVLGLDDIHRAWRTFVLR
VRAQDPPPELYFVKVDVTGAYDTIPQDRLTEVIASIIKPQNTYCVRRYAVV
QKAAHGHVRKAFKSHVSTLTDLQPYMRQFVAHLQETSPLRDAVVIEQSS
SLNEASSGLFDVFLRFMCHHAVRIRGKSYVQCQGIPQGSILSTLLCSLCYG
DMENKLFAGIRRDGLLLRLVDDFLLVTPHLTHAKTFLRTLVRGVPEYGCV
VNLRKTVVNFPVEDEALGGTAFVQMPAHGLFPWCGLLLDTRTLEVQSDY
SSYARTSIRASLTFNRGFKAGRNMRRKLFGVLRLKCHSLFLDLQVNSLQT
VCTNIYKILLLQAYRFHACVLQLPFHQQVWKNPTFFLRVISDTASLCYSIL
KAKNAGMSLGAKGAAGPLPSEAVQWLCHQAFLLKLTRHRVTYVPLLGS
LRTAQTQLSRKLPGTTLTALEAAANPALPSDFKTILD

The catalytic subunit protein of human telomerase has also been referred to as “hEST2” (Meyerson et al., 1997, Cell 90:785), “hTCS1” (Kilian et al., 1997, Hum. Mol. Genet. 6:2011), “TP2” (Harrington et al., 1997, Genes Dev. 11:3109), and “hTERT” (e.g., Greider, 1998, Curr. Biol 8:R178-R181). Human TERT is also described in U.S. patent application Ser. Nos. 08/846,017, 08/844,419, and 08/724,643. The RNA component of human telomerase (hTR) has also been characterized (see U.S. Pat. No. 5,583,016). All of the aforementioned applications and publications are incorporated by reference herein in their entirety. The sequence of a full-length wild type hTR has been deposited in GenBank (Accession No. U85256/NR_{—}001566). The sequence of Homo sapiens telomerase (hTR) is shown below and comprises SEQ ID NO: 8 (nucleotide).

| SEQ ID NO: 8 | |
|---|---|
| | gggttgcgga gggtgggcct gggaggggtg gtggccattt tttgtctaac cctaactgag |
| 61 | aagggcgtag gcgccgtgct tttgctcccc gcgcgctgtt tttctcgctg actttcagcg |
| 121 | ggcggaaaag cctcggcctg ccgccttcca ccgttcattc tagagcaaac aaaaaatgtc |
| 181 | agctgctggc ccgttcgccc ctcccgggga cctgcggcgg gtcgcctgcc cagcccccga |
| 241 | accccgcctg gaggccgcgg tcggcccggg gcttctccgg aggcacccac tgccaccgcg |
| 301 | aagagttggg ctctgtcagc cgcgggtctc tcgggggcga gggcgaggtt caggcctttc |
| 361 | aggccgcagg aagaggaacg gagcgagtcc ccgcgcgcgg cgcgattccc tgagctgtgg |
| 421 | gacgtgcacc caggactcgg ctcacacatg c |

Human TERT is of interest because telomerase activity in human cells and other mammalian cells correlates with cell proliferative capacity, cell immortality, and the development of a neoplastic phenotype. Thus, hTERT polypeptides, including the hTERT variants described herein, and polynucleotides encoding hTERT polypeptides, are used, inter alia, for conferring a telomerase activity (e.g., telomerase catalytic activity, infra) in a telomerase-negative cell such as a cell from a human, a mammal, a vertebrate, or other eukaryote (see, e.g., Bodnar et al., 1998, Science 279:349 and U.S. Pat. Nos. 6,475,789 and 6,166,178). Variants that lack at least one hTERT activity (e.g., telomerase catalytic activity) are used, inter alia, to inhibit telomerase activity in a cell (e.g., by acting as “dominant negative mutants”). The hTERT variants and polynucleotides encoding them, as described herein, are similarly useful in screening assays for identifying agents that modulate telomerase activity.

The invention features methods of diagnosing telomere-associated diseases or disorders in a subject.

The development of genetic detection, e.g. diagnostic methods, for example methods for the early diagnosis of telomere associated diseases or disorders, would allow for timely therapeutic intervention. In certain cases, timely intervention would allow therapy before further damage or death to the subject occurs. Furthermore, the identification of genetic alterations associated with telomere associated diseases or disorders, and the understanding of the role that they play in the development of the pathology are important for new and improved therapeutic strategies.

The invention features methods of diagnosing a telomere-associated disease or disorder in a subject, comprising providing a sample from the subject; and analyzing all or part of a nucleic acid sequence corresponding to one or more of the telomere genes in the sample for the presence of one or more alterations, wherein the presence of at least one alteration is indicative of an increased risk of the presence of a telomere-associated disease or disorder.

The invention also features methods of diagnosing a presence or a predisposition to a telomere-associated disease or disorder in a subject, comprising providing a sample from the subject, and analyzing all or part of a nucleic acid sequence corresponding to one or more telomere genes in the sample for the presence of one or more alterations in the telomere genes, wherein the analyzing comprises amplifying all or part of the nucleic acid sequence of the telomere genes prior to detection, said amplifying being performed with at least one primer consisting of the sequence of any one of SEQ ID NOs: 11-42, wherein the presence of at least one alteration is indicative of an increased risk for the presence or predisposition to a telomere-associated disease or disorder.

The telomere associated gene can be any one or more genes that are associated with the telomere. It is to be understood by one of skill in the art that telomeres are not themselves genes, but the invention is directed to any gene that is associated with a telomere. In certain examples, the telomere associated gene is selected from hTERT (telomerase reverse transcriptase) and hTR (telomerase RNA).

In certain preferred embodiments of the invention, the alteration in the nucleic acid occurs in the telomerase essential N-terminal domain of SEQ ID NO: 7 (hTERT). The sequence of Homo sapiens telomerase reverse transcriptase (hTERT) is shown above, and comprises SEQ ID NO: 7 (nucleotide) and SEQ ID NO: 9 (amino acid).

In other certain embodiments, the alteration in the nucleic acid occurs in the C-terminal domain of SEQ ID NO: 7.

In certain examples, the alteration in the nucleic acid results in a polypeptide comprising an amino acid sequence that differs from SEQ ID NO: 7 (hTERT) by one or more alterations selected from the group consisting of: Leu55Gln, Thr111Met, codon 112delC, intervening sequence between exons IVS1+1G to A, and IVS9-2A to C.

The RNA component of human telomerase (hTR) consists of 451 nucleotides with the 5′ half folding into a highly conserved catalytic core comprising the template region and an adjacent pseudoknot domain (nucleotides 1-208). Gavory et al. (Biochemistry 2006; November 7 45(44): 13304), incorporated by reference in its entirety herein, use fluorescence resonance energy transfer (FRET) between fluorescently labelled peptide nucleic acids, hybridized to defined single stranded regions of full length hTR, to evaluate long-range distances and using molecular modeling, the distance constraints derived by FRET were subsequently used, together with the known secondary structure, to generate a 3D model of the catalytic core of hTR. to determine the 3-dimensional structure of hTR. Although there is considerable variability in the sequence and length of telomerase RNAs across species, there are numerous structural features that appear to be conserved (Chen J L, Greider C W. An emerging consensus for telomerase RNA structure. Proc. Natl. Acad. Sci. U.S.A. 2004; 101:14683-14684; Lin J, Ly H, Hussain A, Abraham M, Pearl S, Tzfati Y, Parslow T G, Blackburn E H. A universal telomerase RNA core structure includes structured motifs required for binding the telomerase reverse transcriptase protein. Proc. Natl. Acad. Sci. U.S.A. 2004; 101:14713-14718). Mutagenesis analyses have demonstrated that the domain-spanning nucleotides 1-208 of hTR are critical for catalytic activity (Autexier C, Pruzan R, Funk W D, Greider C W. Reconstitution of human telomerase activity and identification of a minimal functional region of the human telomerase RNA. EMBO J. 1996; 15:5928-5935), whereas the remaining RNA (nt 209-451) is considered to be primarily involved in protein-RNA interactions (CR4-CR5) and biogenesis of hTR in vivo (box H/ACA domain) (Bachand F, Triki F, Autexier C. Human telomerase RNA-protein interactions. Nucleic Acids Res. 2001; 29:3385-3393; Mitchell J R, Cheng J, Collins K. A box H/ACA small nucleolar RNA-like domain at the human telomerase RNA 3′ end. Mol. Cell. Biol. 1999; 19:567-576). The template region of the RNA comprises a short sequence complementary to the telomeric repeat and serves a catalytic role directing the sequence of newly synthesized telomeric repeats (Morin G B. The human telomere terminal transferase enzyme is a ribonucleoprotein that synthesizes TTAGGG repeats. Cell. 1989; 59:521-529). The P1a and P1b helices are formed by long-range base-pairing interactions that enclose the template and pseudoknot. In particular, helix P1b is responsible for defining the 5′ boundary during repeat addition (Chen J L, Greider C W. Template boundary definition in mammalian telomerase. Genes Dev. 2003; 17:2747-2752). The pseudoknot domain comprises the P3 helix, the structural helix P2b and the two loops J2a/3 and J2b/3. Two additional structural helices P2a1 and P2a present in hTR appear not to be conserved. Functional analysis of the pseudoknot, and mutated derivatives, has demonstrated that stable base pairing within a static P3 helix is a critical requirement (Chen J L, Greider C W. Functional analysis of the pseudoknot structure in human telomerase RNA. Proc. Natl. Acad. Sci. U.S.A. 2005; 102:8080-8085). It has been proposed that both the pseudoknot and template elements are recognized by the hTERT protein component (Chen J L, Greider C W. An emerging consensus for telomerase RNA structure. Proc. Natl. Acad. Sci. U.S.A. 2004; 101:14683-14684). It has been shown in vitro that hTR has the ability to dimerize via RNA interaction sites that include the P3 helix (Ly H, Xu L F, Rivera M A, Parslow T G, Blackburn E H. A role for a novel ‘trans-pseudoknot’ RNA-RNA interaction in the functional dimerization of human telomerase. Genes Dev. 2003; 17:1078-1083), the P1 helix (Moriarty T J, Marie-Egyptienne D T, Autexier C. Functional organization of repeat addition processivity and DNA synthesis determinants in the human telomerase multimer. Mol. Cell. Biol. 2004; 24:3720-3733), and the J7b/8a loop (Marie-Egyptienne D T, Cerone M A, Londono-Vallejo J A, Autexier C. A human-Tetrahymena pseudoknot chimeric telomerase RNA reconstitutes a nonprocessive enzyme in vitro that is defective in telomere elongation. Nucleic Acids Res. 2005; 33:5446-5457).

In certain embodiments, the pseudoknot domain comprises SEQ ID NO: 8. In certain examples, the alteration in the nucleic acid occurs in the pseudoknot domain of SEQ ID NO: 8 (hTR). In certain examples, the alteration in the nucleic acid results in a polypeptide comprising an amino acid sequence that differs from SEQ ID NO: 8 (hTR) by an alteration at residue 98 comprising a G to A substitution.

It is possible that any the substitution may comprise a conservative amino acid substitution. For example, the substitution at amino acid 98 of SEQ ID NO: 8 may be a substitution of G to A, S or T.

As used herein, “conservative substitution,” refers to substitution of amino acids with other amino acids having similar properties (e.g., acidic, basic, positively or negatively charged, polar or non-polar). The following six groups each contain amino acids that are conservative substitutions for one another: 1) alanine (A), serine (S), threonine (T); 2) aspartic acid (D), glutamic acid (E); 3) asparagine (N), glutamine (Q); 4) arginine (R), lysine (K); 5) isoleucine (I), leucine (L), methionine (M), valine (V); and 6) phenylalanine (F), tyrosine (Y), tryptophan (W) (see also, Creighton, 1984, PROTEINS, W. H. Freeman and Company).

In certain preferred embodiments, the one or more alterations is selected from the group consisting of SEQ ID NO: 1, SEQ ID NO: 2, SEQ ID NO: 3, SEQ ID NO: 4, SEQ ID NO: 5, and SEQ ID NO: 6 as follows:

SEQ ID Nos 1-5 comprise the sequence of hTERT (SEQ ID NO:7) with the below alterations:
SEQ ID NO: 1 (Leu55Gln)
SEQ ID NO: 2 (Thr111Met)
SEQ ID NO: 3 (codon 112delC)
SEQ ID NO: 4 (intervening sequence between exons IVS1+1G to A)
SEQ ID NO: 5 (IVS9-2A to C)
   SEQ ID No 6 comprises the sequence of hTR (SEQ ID NO:8) with the below alteration:
SEQ ID NO: 6 (G98A)

It is readily understood by one of skill in the art that any one alteration in a nucleic acid corresponding to a telomere associated gene, or any one or more, e.g. a combination of alterations in nucleic acids corresponding to telomere associated genes, are envisioned in any of the methods as described herein.

The invention features methods of detecting the presence or absence of an alteration in a nucleic acid in a sample. The methods comprise analyzing all or part of a nucleic acid corresponding to a telomere associated gene in the sample and a control sample to determine whether one or more alterations are present in the sample nucleic acid, and determining the presence or absence of one or more alterations in the sample nucleic acid compared to the control sample nucleic acid.

Certain preferred diagnostic methods of the invention involve the detection of telomerase sequences, in certain embodiments a telomerase associated gene, in cell or tissue samples. The cell or tissue samples may be taken from subjects that are suspected to be at risk for a telomere associated disease or disorder. Such methods will typically involve binding a labelled probe or primer to an RNA component sequence under conditions such that only complementary sequences hybridize to one another. Detection of labelled material bound to RNA in the sample will correlate with the presence of telomerase activity and the presence of cancer cells. It is possible that some cells may express the RNA component of telomerase but remain telomerase-negative due to lack of expression of the protein components of telomerase. In order to detect the presence of telomerase activity in such cells, a method can be used to first isolate protein and then determine whether the protein fraction contains the telomerase RNA component, which would indicate telomerase activity. The diagnostic methods of the invention may be especially useful in detecting the presence of telomerase activity in tissue biopsies and histological sections in which the method is carried out in situ, typically after amplification of telomerase RNA component using PCR primers, such as those indicated herein.

Method of diagnosis of certain diseases or disorders have clinical implications. For example, subjects with alterations in one or more certain telomere genes may be categorized according to response, or failure to respond to certain therapies, for instance in an example of aplastic anemia, patients who carry either hTERT or hTR mutations are unlikely to have a response to immunosuppression and may be good candidates for investigational clinical trials. Thus, the presence of a diagnostic genetic test gives patients at risk and their clinicians a chance to consider early screening and evaluation tailored to identification of complications of dyskeratosis congenita. Patients with dyskeratosis congenita, especially those with severe forms, have a predisposition to cancers of the skin, hematopoietic system, and oral mucosa.

In certain examples, the presence of one or more alterations in the telomere associated gene is indicative a disease or disorder selected from dyskeratosis congenita, hematopoietic defects, idiopathic pulmonary fibrosis, idiopathic interstitial pneumonia, and cryptogenic liver cirrhosis.

The methods of the invention are particularly suited to determining disease risk. Thus, the invention features in certain examples methods for determining the risk for a subject to develop a telomere-associated disease or disorder, comprising detecting the presence or absence of an alteration in a telomere gene in a test sample obtained from the subject, comprising analyzing all or a part of a nucleic acid corresponding to a telomere gene in the sample and a control sample to determine whether at least one alteration is present in the test sample nucleic acid wherein the control sample comprises a telomere gene; and determining the presence or absence of one or more alterations in the telomere gene in the test sample compared to the telomere gene in the control sample nucleic acids, wherein the presence of one or more alterations in the telomere gene in the test sample compared to the control correlates with increased risk to develop a telomere associated disease or disorder.

Telomere length can also be used in combination with the above method of nucleic acid detection, or on its own, in methods of diagnosis. In certain examples, the invention features methods of diagnosing a presence or a predisposition to a telomere-associated disease or disorder in a subject, comprising providing a sample from a the subject; and determining the average length of telomeres in the sample compared to a control, wherein a shorter average telomere length is indicative of an increased risk for the presence or predisposition to a telomere-associated disease or disorder.

Thus, the invention features methods of diagnosing a telomere-associated disease or disorder in a subject, comprising providing a sample from a the subject, and determining the average telomere length in the sample compared to a control, wherein a shorter average telomere length is indicative of an increased risk of the presence of a telomere-associated disease or disorder.

The invention also features methods for determining the risk for a subject to develop a telomere-associated disease or disorder, comprising determining the average length of telomeres in a test sample obtained from the subject, comprising providing a sample from a the subject; and determining the average length of telomeres in the sample compared to a control, wherein a shorter average telomere length is indicative of an increased risk to develop a telomere associated disease or disorder.

Telomeres are repetitive DNA sequences located at the termini of linear chromosomes of most eukaryotic organisms. Telomeres compensate for incomplete semi-conservative DNA replication at chromosomal ends. The protection against homologous recombination and non-homologous end joining constitutes the capping role of telomeres.

Telomere length varies among species, from ˜300-600 by in yeast (Shampay et al., 1984) to many kilobases in humans, and usually is comprised of arrays of 6-8 by long G-rich repeats. Eukaryotic telomeres normally terminate with 3′ ssDNA overhang which is essential for telomere maintenance and capping. Multiple proteins binding single-and double-stranded telomere DNA have been identified (Blackburn, 2001; Smogorzewska and de Lange, 2004; Cech, 2004; De Lange et al., 2005; Kota and Runge, 1999). As discussed below, these functions in both telomere maintenance and capping.

Human somatic cells lacking telomerase gradually lose telomeric sequences as a result of incomplete replication (Counter et al., 1992). As human telomeres grow shorter, eventually cells reach the limit of their replicative capacity and progress into senescence. Telomere shortening in humans can induce replicative senescence which blocks cell division.

Telomere length can be determined by a number of different methods that are well known in the art.

In certain embodiment, telomere length is determined by a method selected from the group comprising terminal restriction fragment analysis, fluorescent in situ hybridization, flow cytometry, and quantitative polymerase chain reaction (PCR).

Average telomere length can be determined in any cell of interest, and in particular in leukocytes or granulocytes.

In certain examples, average telomere length is used as a surrogate marker for a telomere-associated disease or disorder.

When the average telomere length is determined in a sample subject compared to a control subject, in certain embodiments the telomere length in the sample from a subject is between 0.3-2.7 kB less than a sample from a control. In certain cases, the average telomere length can be used as a predictive measure of telomerase alteration, and a predictive measure of a disease state, or a risk of developing a disease or condition, or in the tailoring of a course of therapy. For instance, the onset of therapy or the agressiveness with which therapy is pursued may be altered dependent upon determination of telomere length, or determination of telomere length over time.

In certain cases, an average telomere length between is predictive of a telomerase alteration.

In certain preferred examples, the presence of an alteration is detected by comparison with the corresponding nonmutated natural sequence. By nonmutated natural sequence is meant the wild type sequence, in certain preferred embodiments SEQ ID NO; 7 or SEQ ID NO: 8.

The nucleic acid alteration in the telomerase gene can be detected by screening for any one of a number of alterations. For example, the nucleic acid alteration in the telomerase gene is detected by screening for a deletion mutation. The nucleic acid alteration in the telomerase gene is detected by screening for a point mutation. The nucleic acid alteration in the telomerase gene is detected by screening for an insertion. The nucleic acid alteration in the telomerase gene is detected by screening for an inversion. The nucleic acid alteration in the telomerase gene is detected by screening for a missense mutation. The nucleic acid alteration in the telomerase gene is detected by fluorescence in situ hybridization of a telomere gene or part thereof with nucleic acid probes which comprise the telomere gene.

In certain cases, the alteration is a change in copy number, and can be detected by fluorescence in situ hybridization.

The methods as described herein may comprise an additional step of determining telomere activity. A number of methods to determine telomere activity are known in the art.

Diseases/Disorders

The present invention features methods of detection, diagnosing a presence or a predisposition to, or determining the risk for a subject to develop telomere-associated diseases or disorders. A telomere associated disease or disorder can be any disease or disorder associated with a telomere. For example, the telomere associated disease or disorder may be associated with a defect or alteration in telomerase. In certain examples, germline mutations in the telomerase components hTERT or hTR may be associated with telomere associated diseases or disorders, for example a complex phenotype of dyskeratosis congenita in humans. The alteration may be heterozygous or homozygous.

In certain preferred examples, the telomere-associated disease or disorder is selected from, but not limited to, dyskeratosis congenita, hematopoietic defects, idiopathic pulmonary fibrosis, idiopathic interstitial pneumonia, and cryptogenic liver cirrhosis.

### Samples

The subject of the invention as described herein may be any vertebrate. In certain examples, the subject is a mammal, and in particular, a human.

The samples used in the invention herein may be any sample, e.g. any biological sample. In preferred methods of the invention, the sample is peripheral blood; however the sample can include, but not be limited to, tissue samples such as biopsy samples and biological fluids such as whole blood, sputum, buccal swabs, urine and semen samples, bacterial cultures, soil samples, food samples, any other cell type or sample that contains DNA or other nucleic acid etc. In certain preferred examples, genomic DNA is isolated from peripheral blood. The target nucleic acid may be of any origin, including animal, plant or microbiological (e.g., viral, prokaryotic, and eukaryotic organisms, including bacterial, protozoal, and fungal, etc.) depending on the particular purpose of the test. Examples include surgical specimens, specimens used for medical diagnostics, specimens used for genetic testing, environmental specimens, food specimens, dental specimens and veterinary specimens. Depending on the sample, it may only be necessary to lyse the cells, denature the DNA and hybridize to the probes. If required, genomic DNA may be purified from cells using commercially available kits. In some instances, for example, when RNA is present, the sample may need to be purified by techniques known in the art, so that the RNA does not interfere with detection.

The sample may be processed or purified prior to carrying out the instant method in accordance with techniques known or apparent to those skilled in the art; and nucleic acids therein may be digested, fragmented, and/or amplified prior to carrying out the instant method, if so desired. Preferably the sample contains nucleic acid in a sufficient quantity so that amplification is not required; however, amplification may be utilized if desired to improve detection capability. If amplification is required, probes may be used, and an amplification method, such as PCR, employed.

### Primers

The invention features primers that can be used in the methods of the invention as described herein. Any primer sequence can be designed using design methods well known in the art. For instance, publicly available resources such as Primer3 (publicly available on the world wide web at frodo.wi.mit.edu/) can be used to design virtually any primer sequence. Other commercially available resources are readily available to one of skill in the art.

In certain examples, primers are designed to span exons where a mutation is predicted to alter splicing. The primers are advantageously used to detect in a subject a genetic risk for developing a telomere-associated disease or disorder.

In certain preferred examples, the primers are selected from SEQ ID NOs: 11-42 listed below. SEQ ID Nos 11-27 represent forward primers for hTERT. SEQ ID Nos: 28-42 comprise reverse primers for hTERT.

| | |
|---|---|
| SEQ ID NO: 11 | gagtttcaggcagcgctgcgtc |
| SEQ ID NO: 12 | caggacgcgtggaccgagtgacc |
| SEQ ID NO: 14 | cttggtgagctggatgtgcggtg |
| SEQ ID NO: 15 | gcctcctgaggggctctctattg |
| SEQ ID NO: 16 | gccggatccactttcctgactgt |
| SEQ ID NO: 17 | gtctgatgtgtggtagctgcag |
| SEQ ID NO: 18 | gtggtcctgcctgtctcagcacc |
| SEQ ID NO: 19 | ggctgaatggtagacgtgtcg |
| SEQ ID NO: 20 | gcacagaattgcacaagctgatg |
| SEQ ID NO: 21 | gcatcctggggctgacattgcc |
| SEQ ID NO: 22 | gtccatggagtgagcacccagc |
| SEQ ID NO: 23 | gatgacacagagtctgactcgc |
| SEQ ID NO: 24 | cacacccacgagcaccgtctg |
| SEQ ID NO: 25 | tgtgactcctcagcctctgtt |
| SEQ ID NO: 26 | gccgctccagcgtcactg |
| SEQ ID NO: 27 | caggtgaaccagcacgtcgtcgc |
| SEQ ID NO: 28 | cttgtcgcctgaggagtagag |
| SEQ ID NO: 29 | gtgaactcgtaagtttatgc |
| SEQ ID NO: 30 | gagccttgcacagaatccacttg |
| SEQ ID NO: 31 | ctcccaaggtccagcagggtgc |
| SEQ ID NO: 32 | cacagacacgactgcattctagac |
| SEQ ID NO: 33 | caacagtctgtccggtcatgag |
| SEQ ID NO: 34 | gcttgccatttccaggcctcgtg |
| SEQ ID NO: 35 | gagcagtcatggtctccagagc |
| SEQ ID NO: 36 | ctgctcttgcggatccagcacc |
| SEQ ID NO: 37 | cagccgggcacaggctccact |
| SEQ ID NO: 38 | cagtcaccatcagccttgcag |
| SEQ ID NO: 39 | cgcgaacagaactgtgcacag |
| SEQ ID NO: 40 | acagggcgttcaaggatgacc |
| SEQ ID NO: 41 | cagcagcatctgaggctgctcg |
| SEQ ID NO: 42 | agtgcacattaggattca |

The invention features primer sets for detecting in a subject a genetic risk for developing a telomere-associated disease or disorder, the primer set having a forward and reverse primer. In certain examples, the forward primer is selected from the group consisting of: SEQ ID NOs: 11-27, and a reverse primer selected from the group consisting of: SEQ ID NOs: 28-42.

### Therapeutics

The methods of the invention as described herein can further comprise the administration of a therapeutic agent. For example, in certain cases, the presence of shortened telomeres in a subject, for example in a subject that does not have one or more telomerase mutations, may suggest that telomerase shortening plays a role in disease pathogeneisis, and that strategies aimed at preventing cell death, or local responses to it, may have an impact in attenuating the course of the disease. Accordingly, the administration of a therapeutic agent is beneficial.

In certain preferred examples, the therapeutic agent prevents cell death, e.g. an anti-apoptotic agent. An anti-apopototic agent can be a chemotherapeutic. In other certain examples, the agent promotes cell proliferation.

The invention also encompasses therapeutic methods. Therapeutic methods of the invention involve the administration of an agent, for example an oligonucleotide that functions to inhibit or stimulate telomerase activity. Advantageously, the oligonucleotide will be active under in vivo physiological conditions and will be stable under those conditions. For example, nucleic acids, e.g. modified nucleic acids, may be useful in imparting such stability, as well as for ensuring delivery of the oligonucleotide to the desired tissue, organ, or cell. Methods useful for delivery of oligonucleotides for therapeutic purposes are described in Inouye et al., U.S. Pat. No. 5,272,065, incorporated herein by reference in its entirety.

While oligonucleotides can be delivered directly as a drug in a suitable pharmaceutical formulation, one can also deliver oligonucleotides using gene therapy and recombinant DNA expression plasmids of the invention. In general, such plasmids will comprise a promoter and, optionally, an enhancer (separate from any contained within the promoter sequences) that serve to drive transcription of an oligoribonucleotide, as well as other regulatory elements that provide for episomal maintenance or chromosomal integration and for high-level transcription, if desired. Adenovirus-based vectors are often used for gene therapy, see PCT patent publication Nos. 94/12650; 94/12649; and 94/12629, incorporated by reference in its entirety herein. Useful promoters for such purposes include the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), and the constitutive CMV promoter. A plasmid useful for gene therapy can comprise other functional elements, such as selectable markers, identification regions, and other genes. Recombinant DNA expression plasmids can also be used to prepare the oligonucleotides of the invention for delivery by means other than by gene therapy, although it may be more economical to make short oligonucleotides by in vitro chemical synthesis.

In related aspects, the invention features pharmaceutical compositions including a therapeutically effective amount of a telomerase inhibitor or telomerase activator of the invention. Pharmaceutical compositions of telomerase inhibitors of the invention include a mutant RNA component of human telomerase, an antisense oligonucleotide or triple helix-forming oligonucleotide that binds the RNA component or the gene for the same of human telomerase, or a ribozyme able to cleave the RNA component of human telomerase, or combinations of the same or other pharmaceuticals in a pharmaceutically acceptable carrier or salt. Other pharmaceutical compositions of the invention comprise a telomerase activator preparation, such as purified human telomerase or mRNA for the protein components of telomerase and the RNA component of telomerase, and are used to treat senescence-related disease. The therapeutic agent can be provided in a formulation suitable for parenteral, nasal, oral, or other mode of administration. See, e.g. PCT patent publication No. 93/23572, incorporated by reference in its entirety herein.

### Kits

Within the scope of the present invention are also included diagnostic kits for the identification, in an individual, of the mutation according to the invention.

The invention features, in certain examples, kits for use in diagnosing a telomere-associated disease or disorder in a subject or diagnosing the presence or a predisposition a telomere-associated disease or disorder in a subject, and instructions for use. The kits can comprise the primer sets as described herein, along with instructions for use.

The kits of the invention can also be used for in vitro diagnostics. Thus, the present invention also relates to a method for the in vitro diagnosis of telomere associated diseases or disorders in a subject, including testing for the presence, in a sample from the subject, of a nucleic acid sequence corresponding to one or more of the telomere genes in the sample for with one or more alterations, according to the invention, wherein the identification of one or more alterations is an indication that the individual is suffering from a telomere associated disease or disorder.

Having now generally described the invention, the same will be more readily understood through reference to the following Examples, which are provided by way of illustration, and are not intended to be limiting of the present invention, unless specified.

### EXAMPLES

A pedigree with autosomal dominant dyskeratosis congenita that carried a null hTERT allele but lacked the typical mucocutaneous features was recently identified. In this kindred, pulmonary fibrosis was dominantly transmitted, and was the only manifestation of disease in one mutation carrier. The clinical presentation and pattern of fibrosis in this subject were typical of the idiopathic form of the disease. Since familial idiopathic pulmonary fibrosis is also dominantly inherited, it was hypothesized that telomere shortening causes this disease and that mutations in telomerase may contribute to it.

### Example 1

### Mutations Affecting Telomerase Components

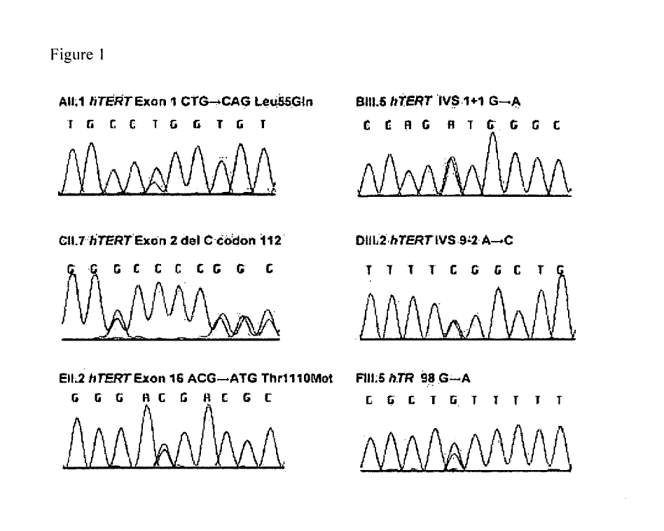

In a first example, 73 probands were screened. Of the 73 probands who were screened, 6 (8%) had heterozygous mutations in hTERT or hTR. Five probands had mutations in hTERT (two missense, two splice junction, and one frameshift), and one proband had a mutation in hTR (Table 1, below, and FIG. 1). Table 1 shows mutations in telomerase and associated clinical features of the six probands. FIG. 1 shows chromatograms of telomerase mutations in probands from Families A-F. None of the hTERT mutations were present in 623 unaffected subjects, as determined in other studies (23, 31). Of these subjects, 140 described themselves as white, with the rest describing themselves as black, Hispanic, or Asian. The hTR mutation was also absent in 194 healthy controls. Of these subjects, 123 described themselves as white, with the remaining subjects describing themselves as black, Hispanic, or Asian (22).

TABLE 1

| | | | Age | | | | | | |
|---|---|---|---|---|---|---|---|---|---|
| | | | At | At Time | | | Pulmonary Function | | |
| Proband | | | Onset | of Study | Presenting | Smoking | TLC | FVC | DLCO |
| No. | Mutation | Sex | yr | | Symptom | History | liters (% of predicted value) | | |
| AII.1 | hTERT | M | 77 | 81 | Dyspnea | None | 4.45 (68) | 3.02 (68) | 14.2 (76) |
| | CTG→CAG | | | | | | | | |
| | Leu55Gln | | | | | | | | |
| BIII.5 | hTERT | M | 58 | 67† | Cough | None | 3.17 (44) | 2.10 (44) | 12.5 (49) |
| | IVS1 + 1 G→A | | | | | | | | |
| CII.7 | hTERT | M | 58 | 61 | Dyspnea | 30 pack-years | 5.28 (69) | 3.55 (66) | 12.8 (47) |
| | codon 112 | | | | | | | | |
| | del C | | | | | | | | |
| DIII.2 | hTERT | F | 48 | 49† | Dyspnea | 32 pack-years | NA | 1.31 (43) | NA |
| | IVS9.2 A→C | | | | | | | | |
| EII.2 | hTERT | F | 68 | 76 | Dyspnea | None | 3.25 (68) | 1.69 (47) | 12.5 (54) |
| | ACG→ATG | | | | | | | | |
| | Thr1110Met | | | | | | | | |
| FIII.5 | hTR | F | 60 | 66† | Dyspnea | None | 2.48 (51) | 1.2 (45) | 7.07 (32) |
| | 98 G→A | | | | | | | | |

| | | Complete Blood Count | | |
|---|---|---|---|---|
| Proband | | WBC | Hgb | Platelet |
| No. | Findings on Lung Biopsy | per mm³ | g/dl | per mm² |
| AII.1 | Usual interstitial pneumonia | 5,500 | 14.0 | 206,000 |
| BIII.5 | Usual interstitial pneumonia | 8,800 | 14.1 | 282,000 |
| CII.7 | Usual interstitial pneumonia | 8,800 | 16.2 | 201,000 |
| DIII.2 | Idiopathic interstitial pneumonia | 10,800 | 13.8 | 235,000 |
| EII.2 | Usual interstitial pneumonia | 9,500 | 15.5 | 317,000 |
| FIII.5 | Usual interstitial pneumonia | 6,800 | 12.8 | 218,000 |

* Identifiers for all probands refer to subjects shown in FIG. 1.
TLC denotes total lung capacity,
FVC forced vital capacity,
DLCO carbon monoxide diffusing capacity,
WBC white cells,
Hgb hemoglobin.
IVS intervening sequence between exons (intronic), and
NA not available.
†The age at the time of death is shown.

### Example 2

### Mutations Associated with Disease and Short Telomere

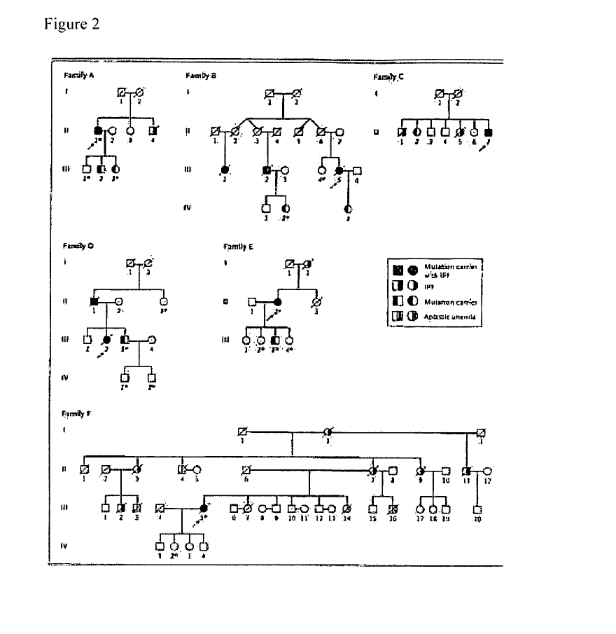

To determine whether telomerase mutations segregated with idiopathic pulmonary fibrosis in families, the pedigrees were examined. The pattern of inheritance was consistent with autosomal dominant inheritance of the disease, as shown in FIG. 2. In FIG. 2, arrows point to the proband in each family, and bold italic numbers indicate subjects for whom DNA was available for sequencing. Subjects in whom telomere length was measured are indicated by asterisks. Mutation status is indicated by the symbols shown in the key, with squares indicating male sex and circles indicating female sex. Deceased family members are indicated by slashes through the symbols. In Family D, Subject DII.1 is an obligate carrier, given that two of his children carry the mutation and the mother does not. A total of 19 subjects with confirmed idiopathic pulmonary fibrosis are included among the six families shown. The seven asymptomatic carriers in younger generations were on average 11 years younger than the probands at the time of diagnosis: 40, 44, 46, 50, 52, 55, and 68 years of age. This observation is consistent with the variable penetrance associated with familial idiopathic pulmonary fibrosis and also suggests that the onset of disease may be age dependent. In Family F, three subjects had aplastic anemia, and Subject FIII.16 died from acute myeloid leukemia, probably in the setting of aplastic anemia. IPF denotes idiopathic pulmonary fibrosis. The mutant allele was present in affected subjects and was generally absent in asymptomatic subjects of the same generation. Mutation carriers were identified who did not have symptoms of the disease.

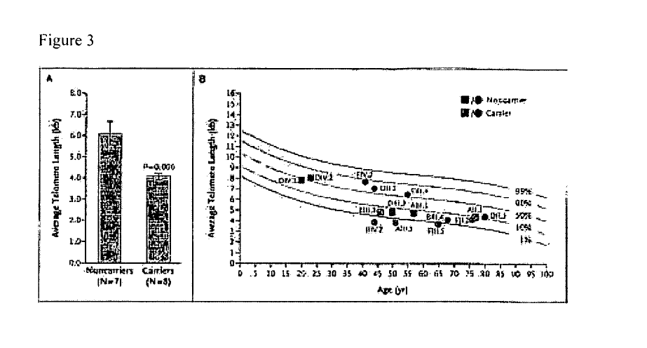

To assess whether mutant telomerase is associated with short telomeres, telomere length was measured in lymphocytes. The average telomere length was significantly less in the probands and asymptomatic mutation carriers than in their relatives who did not carry the mutation (P=0.006), shown in FIG. 3A. In FIG. 3, Panel A shows the average length of telomeres in lymphocytes in eight carriers and seven non-carriers of the genetic mutation, and Panel B shows telomere length as a function of age. The three oldest mutation carriers are the pro-bands in Family A, Family E, and Family F. The 12 other subjects who were examined are indicated in FIG. 1 by an asterisk. Identifiers refer to subjects from the pedigrees in FIG. 1. I bars represent standard errors. Telomere lengths in mutation carriers were significantly less than the median value for their age (P=0.018 by the Wilcoxon signed-rank test), whereas telomere lengths in non-carriers did not differ significantly from the median for their age (P=0.575). A comparison of the telomere length in mutation carriers with that in 400 healthy controls, according to age, (23) showed that mutation carriers fell below the 10th percentile of the controls (P=0.018), whereas their relatives who were non-carriers clustered near the median (P=0.575) (FIG. 3B). Mutant telomerase was therefore associated with short telomeres.

### Example 3

### Impaired Activity of Mutant Telomerase

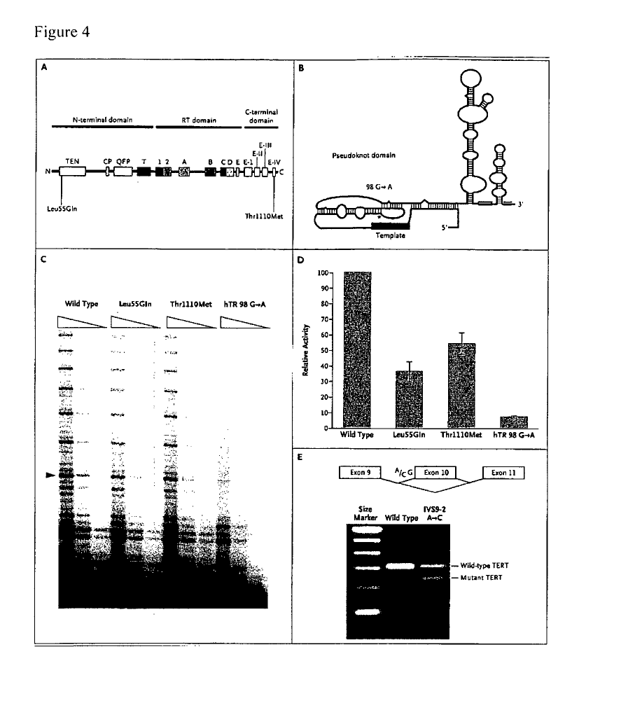

Next, the consequences of hTERT and hTR mutations on telomerase function were examined. The results are shown in FIG. 4, which show the biochemical consequences of telomerase mutations in probands.

First, the two missense mutations in hTERT, glutamine replacing leucine at residue 55 (Leu55Gln) and methionine replacing threonine at residue 1110 (Thr1110Met) were examined. The Leu55Gln substitution identified in the proband of Family A is in a highly conserved region of the N-terminal; an amino acid substitution of Leu55 may alter telomerase RNA binding and thus the catalytic efficiency of telomerase (32). The Thr1110 residue is also highly conserved and lies in the C-terminal of hTERT, a domain that is thought to mediate recruitment of telomerase to the telomere (33). Panel A shows conserved domains of hTERT with missense mutations, as indicated. Colors indicate conserved domains in hTERT shared with other reverse transcriptases. The Leu55Gln mutation lies in the telomerase essential N-terminal (TEN) domain, and Thr1110Met is in one of four conserved C-terminal domains. Panel B shows the secondary structure of hTR, with the site of the mutation indicated by an asterisk. The 98 G-A substitution lies in a critical helix of the pseudoknot domain, which contains the telomere template and is responsible for binding to TERT. Panel C shows the telomerase activity of mutant hTERT and hTR alleles, as measured by the direct assay and the intensity and pattern of the repetitive ladder. Panel D shows the quantitation of telomerase activity at the second major band, as indicated by the arrowhead in Panel C. Mean activity was calculated on the basis of three to five experiments; the I bars represent standard errors. Panel E shows the results of an RT-PCR assay across exons 9 through 11 from a subject with an hTERT 9-2 A-4C mutation, indicating that the heterozygous mutation at this consensus splice junction leads to the skipping of exon (10). As a result, the mutant TERT lacks the essential motif C of the reverse-transcriptase domain.

Both mutant versions of hTERT (Leu55Gln and Thr1110Met) had impaired activity, as compared with the wild-type enzyme (FIG. 4C and 4D). Since heterozygous mutations sometimes interfere with the function of the wild-type allele, the telomerase activity of a mixture of wild-type and mutant versions of the enzyme was assayed, and no dominant negative effect was observed (data not shown). The effect of the hTR 98 G-*A substitution (observed in the proband of Family F) on telomerase activity was also examined. This mutation is predicted to impair base pairing in a helix in the essential pseudoknot domain of hTR (34). Moreover, since 98G is conserved in telomerase RNA in all vertebrates, a mutation at this site is expected to alter activity (34). When telomerase was reconstituted with the mutant hTR 98A allele, activity was severely impaired, as shown in FIG. 4C and 4D. Next, the potential consequences of the three mutations in hTERT were examined. The deletion of nucleotide C at codon 112 in the proband of Family C leads to a frameshift mutation and is predicted to result in a nonfunctional, truncated protein. Both splice-junction mutations in Family B and Family D occur at consensus sequences that are conserved in 99.9% of all eukaryotic genes and are therefore predicted to alter splicing. The cDNA of primary cells from a subject in Family D who carried the IVS9-2 A-3C mutation and observed skipping of exon 10 but retention of the reading frame was examined (FIG. 4E). According to these findings, obtained by RT-PCR, synthesis of a protein of nearly full length is predicted. However, this mutant TERT is predicted to lack an essential motif (the C motif) in the reverse transcriptase domain and thus to result in a functionally null protein (FIG. 4A).

Clinical Review

The probands were re-examined for the most common features of dyskeratosis congenita. None of the probands had cytopenias (as shown in Table 1, above), and none had any of the classic features of dyskeratosis congenita at the time of diagnosis. To discern whether these six families had hidden cases of dyskeratosis congenita, family members and medical records were required for evidence of aplastic anemia. No cases of aplastic anemia were identified in five of the six families. In Family F, three subjects were identified with aplastic anemia and a fourth subject with probable aplastic anemia (FIG. 2, above). In this family, subjects with a hematopoietic defect died at a younger age (25, 26, 31, and 81 years, with a mean of 41 years) than did those with idiopathic pulmonary fibrosis (76, 70, 63, 57, 60, and 66 years, with a mean of 65 years). The possibility that asymptomatic mutation carriers with short telomeres had cytopenias that reflected early changes of aplastic anemia was also explored. Complete blood counts in members of five of the families were examined—Family A, Family B, Family C, Family D, and Family E—and no abnormalities were found.

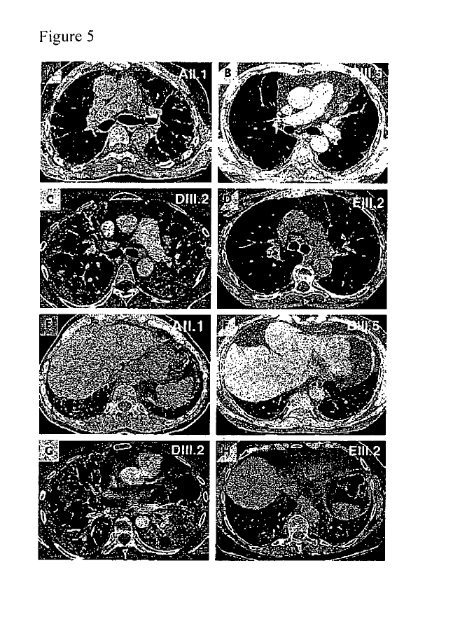

To assess whether the pulmonary fibrosis in the probands could be differentiated from other cases of idiopathic pulmonary fibrosis, the clinical data was reviewed. The presentation, age at onset, and findings on computed tomography were indistinguishable from those of other cases of the disease (Table 1, above, and FIG. 5). In FIG. 5, subject numbers are shown in the upper right-hand corners of the panels. In all four probands, subpleural honeycombing and increased reticular densities are visible in the views at the bases. These changes extend up to the midlung and apexes in some subjects with more advanced stages of disease (e.g., Subject DIII.2). None of these subjects had a response to trials of immunosuppressive therapy. In all cases, the proband had undergone lung biopsy, and five of the six pro-bands had the common lesion of usual interstitial pneumonia. A biopsy specimen obtained from the sixth proband showed idiopathic interstitial pneumonia, not classifiable. Different idiopathic interstitial pneumonia pathological lesions have been described in the same patient, as well as in members of the same family with the disease, underscoring the need for precise molecular characterization.

The results presented herein demonstrate that mutant telomerase is associated with familial idiopathic pulmonary fibrosis, which suggests that the spectrum of disease caused by telomere shortening is more extensive than previously appreciated and that a subgroup of families with pulmonary fibrosis falls on that spectrum. Short dysfunctional telomeres activate a DNA damage response that leads to cell death or cell cycle arrest. This response is manifested clinically as organ failure in tissues of high turnover (bone marrow, skin, and gastrointestinal tract) in patients with, and in an animal model of, dyskeratosis congenita. (14,24) The presence of pulmonary fibrosis in dyskeratosis congenita, along with the presence of telomerase mutations in some families with idiopathic pulmonary fibrosis, suggests that bronchoalveolar epithelium is also constantly replaced and relies on local progenitor reserves that are limited by short telomeres.

On the basis of these findings, one proposal is that the fibrotic lesion in patients with short telomeres is provoked by a loss of alveolar cells rather than by a primary fibrogenic process, such as one that would seem to occur in autoimmune disease associated with lung fibrosis. This view is supported by the fact that misfolded surfactant protein C (present in affected subjects carrying a mutation in the corresponding gene) appears to be toxic to alveolar cells. (4) Therefore, it is possible that in some types of fibrosis, damage of epithelial cells leads to a remodeling response that appears clinically as usual interstitial pneumonia. Taken together, these considerations may explain the lack of success in reversing idiopathic fibrosis with agents that modulate immune or inflammatory signals and support the idea that at least in some cases, strategies aimed at preventing the loss of alveolar cells, or local responses to such cell loss, may have a greater clinical impact.

Although mutations in the essential components of telomerase do not seem to account for a majority of cases of familial pulmonary fibrosis, telomere shortening as a process may still contribute to the pathogenesis. There is evidence that short telomeres, rather than telomerase mutations, correlate with disease in dyskeratosis congenita. In an animal model of dyskeratosis congenita, wild-type mice who inherit short telomeres appear to have an occult genetic disease and display phenotypes similar to those in mice that are heterozygous for mutant telomerase RNA (14). Acquired states that increase tissue turn-over are also associated with short telomeres. One study showed that both current and former smokers had shorter telomeres than did age-matched nonsmokers (36). In addition, there is some evidence that telomeres of the alveolar epithelium in smokers are shorter than those of the alveolar epithelium in nonsmokers (37). It is therefore possible that somatic telomere shortening, caused by conditions that increase cell turnover (e.g., smoking), could contribute to fibrosis. In a study evaluating disease onset in relatives of familial probands with idiopathic pulmonary fibrosis, cigarette smoking and older age were the strongest predictors. 35 Because telomere shortening occurs with aging and can be acquired, it may contribute to the disease pathogenesis even in persons with wild-type telomerase.

The results presented herein have clinical implications. As suggested by the experience in aplastic anemia (23), patients who carry either hTERT or hTR mutations are unlikely to have a response to immunosuppression and may be good candidates for investigational clinical trials. The presence of a diagnostic genetic test gives patients at risk and their clinicians a chance to consider early screening and evaluation tailored to identification of complications of dyskeratosis congenita. Patients with dyskeratosis congenita, especially those with severe forms, have a predisposition to cancers of the skin, hematopoietic system, and oral mucosa. Finally, telomere length may serve as a surrogate marker for the identification of patients at greatest risk for carrying mutant telomerase genes. The data and result presented herein describe 15 subjects, where longer telomeres appeared to predict the absence of a telomerase mutation. Since the consequences of carrying mutant telomerase genes can appear in adulthood as either idiopathic pulmonary fibrosis or aplastic anemia without dyskeratosis, the consideration of such cases as part of a syndrome of telomere shortening may heighten the index of suspicion and facilitate diagnosis.

### Example 4

### Short Telomeres in Idiopathic Pulmonary Fibrosis and Related Disorders

Sporadic IPF patients have short telomeres and carry telomerase mutations even when there is no family history. Rare telomerase mutations can be found in cohorts of patients with idiopathic interstitial pneumonia-idiopathic pulmonary fibrosis even when there is no family history (please see attached manuscript in preparation). This finding underscores the relevance of the genetics of telomere shortening in sporadic IPF.

Idiopathic interstitial pneumonias (IIPs) have a predictable, progressive course that leads to respiratory failure. As their name indicates, the etiology of IIPs is unknown and this has hampered progress in the development of therapies for patients with this disease. Idiopathic pulmonary fibrosis (IPF) is the most common of the IIPs and accounts for more than 70% of all cases (38). It has a characteristic radiographic appearance associated with the pathologic lesion of usual interstitial pneumonia. Age is the biggest risk factor for the development of IIPs with the majority of cases diagnosed after the sixth decade. The factors that contribute to the age-related onset of IIPs are not known. As many as one in five patients with IPF report a family history of the disease establishing genetic factors as a critical contributor to disease risk (39). Histological features of IPF and other IIP subtypes are often present in the same individual and in individuals from a single family, indicating that IIPs share a common etiology (40, 41). Mutations in telomerase components hTERT and hTR underlie inheritance of IPF in 8-15% of individuals with a documented family history (42, 43). In these families, affected individuals have a clinical presentation indistinguishable from sporadic forms of the disease (42). Telomere length, not telomerase mutations, predicts disease onset in syndromes of telomere shortening (44-47). Here the role of telomere shortening and mutations in telomerase components in the pathogenesis of non-familial forms of idiopathic interstitial lung disease. is examined. Short telomeres limit tissue renewal capacity in the lung and germline mutations in telomerase components, hTERT and hTR, underlie inheritance in a subset of families with idiopathic pulmonary fibrosis. Here the hypothesis is examined if telomere shortening contributes to disease risk in sporadic IIPs.

A pedigree was recently described with autosomal dominant dyskeratosis congenita that carried a null mutation in hTERT (44). In this kindred, both pulmonary and liver fibrosis were dominantly inherited and each trait displayed anticipation, an earlier more severe onset of disease with successive generations. The anticipation of the fibrosis phenotypes, along with aplastic anemia, correlated with inheritance of the shortest telomeres across generations and suggested that telomere shortening underlies the predisposition to fibrosis in parenchymal organs and that the fibrosis, similar to aplasia in the marrow, may represent a loss of regenerative capacity (44). Telomere length, and not mutations in telomerase themselves, predict disease onset and severity in models of aplastic anemia and dyskeratosis congenita (49). In these models, wildtype mice who inherit short telomeres, display phenotypes similar to heterozygous mice. Thus, even when telomerase is wildtype, short telomeres limit tissue renewal capacity.

Here, the hypothesis is examined that telomere shortening, in the presence or absence of telomerase mutations, contributes to disease risk in IIP patients who have no family history. We show that similar to familial IPF patients with telomerase mutations, individuals with IIP have short telomeres in both peripheral blood and in the lung. In these patients, there is an increased incidence of other features of dyskeratosis congenita; specifically of cryptogenic liver cirrhosis. The general methodology involved recruiting patients with idiopathic interstitial pneumonia who have no family history and examining telomere length in peripheral blood and alveolar epithelium in the lung. To screen for mutations, genomic DNA was sequenced for hTERT and hTR. Each case was also reviewed for features of a syndrome of telomere shortening.

IIP Patients have Short Telomeres in Peripheral Blood Leukocytes

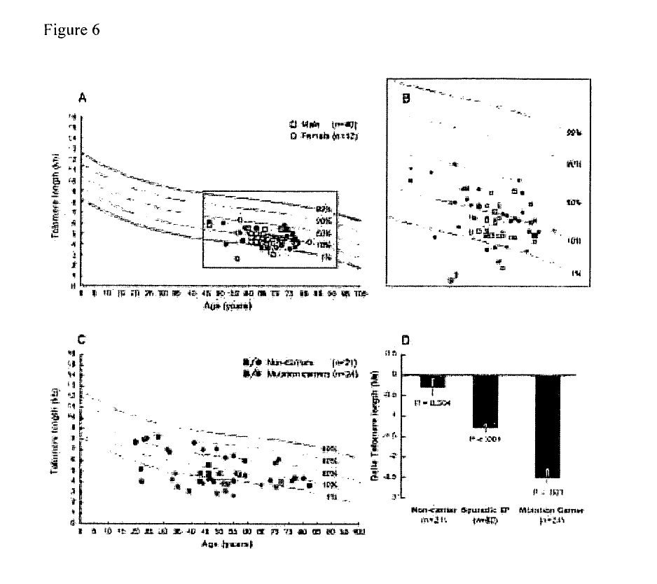

To examine whether individuals with IIP have short telomeres, the telomere length in peripheral blood lymphocytes using flow-FISH. Compared to healthy age-matched controls, (n=400), IIP patients had shorter telomeres (p<0.0001, Wilcoxon signed rank test; FIG. 6A and B). Specifically, 97% (60 of 62) of IIP patients had telomere length less than the median for their age (mean delta telomere −1.3 Kb, range −0.3 to −2.7; probability of random event p<0.0001). To examine whether this effect was cell-type specific, telomere length was examines in granulocytes from the same patients and found a similar trend. Differences in telomere length were not detected within the cohort by gender (p=0.30, multivariate regression analysis adjusting for age), smoking status (p=0.50). These data suggested that individuals with IIP have shorter telomeres in the peripheral blood than healthy age-matched controls.

Taken together, the data presented herein demonstrates that as a group, idiopathic interstitial pneumonia patients had lymphocyte telomeres significantly shorter than age-matched controls. In a subset (10%), sporadic IIP patients had telomere length below the first percentile for their age, a range highly predictive of a germline mutation in telomerase. Similar to familial cases with known mutations, sporadic IPF patients also had telomere shortening in alveolar epithelial cells. Although telomerase mutations were rare, identified in 1 of 100 patients, we identified a cluster of individuals with both pulmonary fibrosis and cryptogenic liver cirrhosis, another feature of a syndrome of telomere shortening. The findings establish a role for telomere shortening in IPF and related disorders beyond a subset of families with telomerase mutations and suggest that telomere shortening may be an important contributor to the genetics of this age-related disease.

Short telomeres are a signature in patients with idiopathic interstitial pneumonia and this may account for the age-related onset of this disease. The clustering of cryptogenic liver cirrhosis in patients with idiopathic pulmonary fibrosis suggests that the telomere shortening we identify in our study has clinical consequences and may be an important risk factor of apparently idiopathic progressive fibrotic processes in both the lung and the liver.

Telomere Length is a Surrogate for Mutation Status in Families with Telomerase Mutations

Short telomeres are associated with telomerase mutations in familial IPF and dyskeratosis congenita. To determine whether telomere length can be a surrogate for mutation status, 10 families were examined with known mutations in telomerase components and compared mutation carriers with their relatives who did not carry mutations. FIG. 6 shows the results of these studies, where telomere length in lymphocytes from IIP patients and families with known telomerase mutations are compared to controls. Individuals with mutations in telomerase components had significantly shorter telomeres than non-carriers (p<0.0001, regression analysis adjusting for age; FIG. 6C). Furthermore, mutation carriers had shorter telomeres compared with the median telomere length of age-matched controls (n=24, p=<0.0001, Wilcoxon signed rank test). In comparison, non-carriers had telomere lengths that were not different from the median (n=21, p=0.304; FIG. 6D). Individuals with mutations in telomerase components also had short telomeres in granulocytes. These data suggested that telomere length in peripheral blood can be a useful surrogate of mutation status in relatives of individuals with known telomerase mutations. More specifically, individuals with lymphocyte telomere length greater than the 50th percentile for age never had mutations (100% predictive value). In contrast, individuals with very short lymphocyte telomeres less than the 1^{st} percentile for age who also had short telomeres in granulocytes (less than the 10th percentile), independent of disease status, had a 95% likelihood of carrying the same mutation as the proband in their family. Thus, within these parameters, telomere length is a useful surrogate for predicting mutation status in relatives of probands with known telomerase mutations.

### A Subset of HP Patients has Very Short Telomeres Similar to Mutation Carriers

To further examine the significance of telomere shortening in non-familial IIP patients, their telomere length was compared with individuals with known mutations and their families. Similar to mutation carriers, IIP patients had shorter telomeres than non-carriers (p=X, multivariate regression analysis adjusting for age). The proportion of IIP patients with very short lymphocyte and granulocyte telomeres was compared, and it was found that 10% of patients (6 of 62) had telomere length below the 1st percentile. This is a range highly predictive of the presence of a telomerase mutation in the families that were examined. Thus, similar to telomerase mutation carriers, a subset of IIP patients who have no family history have very short telomeres in a range similar to mutation carriers.

Detectible Telomerase Mutations are Rare in Patients with Sporadic IPF

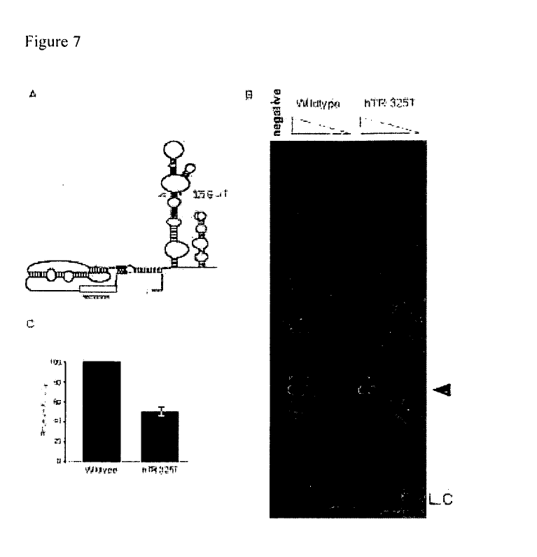

To examine the hypothesis that telomerase mutations underlie the telomere shortening in non-familial IPF, the essential components of telomerase, hTERT and hTR were sequenced in 100 consecutive patients from the Vanderbilt Interstitial Lung Disease clinic, including 62 individuals where telomere length was available. One mutation in hTR was identified, 325G→T, which predicted disruption of a conserved helix of telomerase RNA (30*) (FIG. 7A). This previously undescribed mutation was absent in large series of healthy controls (n=194) (67), was associated with short telomeres and led to a loss of activity as quantitated by the direct assay (FIG. 6B and FIG. 7). Three heterozygous non-synonymous variants of hTERT Ala279Thr (n=8), His412Tyr (n=1) and Ala1062Thr(n=5) were also identified. All three variants have been identified in series of healthy controls (56, 68) and did not show any evidence of compromised telomerase activity when reconstituted in vitro or in cells that lack telomerase (Table 6 and FIG. 6). Thus readily detectible mutations in individuals with sporadic idiopathic lung fibrosis are rare. The presence of individuals with very short telomeres less than the 1st percentile suggests that other genetic mechanisms that lead to telomere shortening play a role.

IPF Patients have Short Telomeres in Alveolar Epithelium

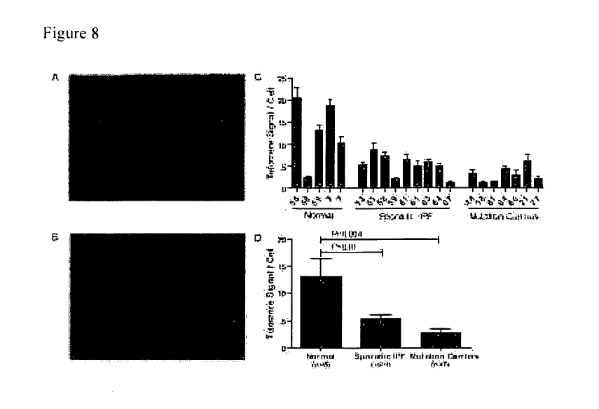

Peripheral blood telomere length may reflect germline telomere length but is also susceptible to states of high turnover in leukocytes. To examine the hypothesis that telomere shortening in IPF reflects a genetic predisposition to having short telomeres in the lung, telomere length was examined in alveolar epithelium using quantitative FISH, as shown in FIG. 8. In situ telomere length was compared from individuals with normal lungs, sporadic IPF, and IPF patients with known telomerase mutations. Alveolar epithelium from individuals with IPF patients who had known telomerase mutations had shorter telomeres than normal controls (p=0.004, two-tailed t-test; FIG. 8). Additionally, individuals without a family history of IPF also had shorter telomeres than healthy controls (p=0.01). These data indicate that, similar to peripheral blood leukocytes, telomeres in alveolar epithelium are shorter in sporadic IPF and that the IPF phenotype, even in the absence of a family history and in the absence of a detectible mutation in telomerase, is associated with short telomeres in the lung.

Cryptogenic Cirrhosis in Patients with Idiopathic Pulmonary Fibrosis

Because of the high prevalence of short telomeres in IIP patients, medical records were retrospectively examined for features of a syndrome of telomere shortening. None of the 100 patients had diagnosed aplastic anemia although the patient with the shortest telomeres in the cohort (FIG. 6B) had chronic unexplained thrombocytopenia associated with macrocytosis, both features of subclinical aplastic anemia. Ten percent of the patients in the cohort had platelet counts less than the normal range. In the absence of a formal work-up, it is difficult to discern but it is interesting to consider the possibility that sub-clinical aplastic anemia may be another manifestation of the short telomeres in IIP patients. Unexplained liver fibrosis is associated with IPF in individuals with dyskeratosis congenita (44,57); the cohort was therefore queried for cases of cryptogenic liver disease. Two patients were identified who were diagnosed with cryptogenic liver cirrhosis after a thorough workup for an etiology (as shown in Table 2, below). Table 2 shows the clinical features of patients with both idiopathic pulmonary fibrosis and cryptogenic liver cirrhosis.

TABLE 2

| | | Age at | | |
|---|---|---|---|---|
| | | Diagnosis, | | |
| | IIP | Presenting | Evidence of | Age at |
| Gender | Diagnosis | Symptom | Cirrhosis | Diagnosis |
| M | IPF | 60, cough | Liver Transplant | 58 |
| M | IPF | 72, cough | Liver Transplant | 65 |
| M | IPF | 65, dyspnea | Portal Hypertension | 65 (d.66) |
| M | IPF | 67, cough | Compensated Cirrhosis | 59 |

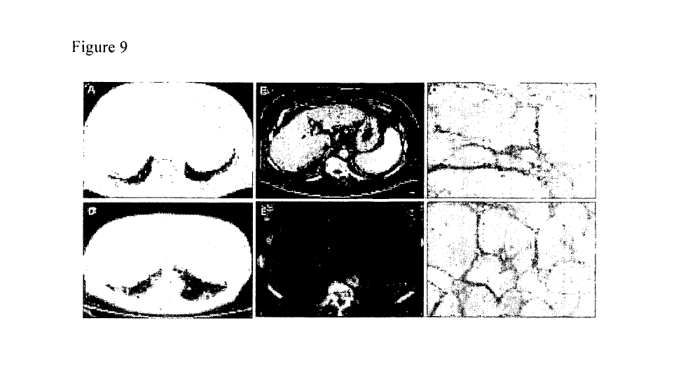

To probe this observation further, the records of the last 50 consecutive IPF patients seen in the Johns Hopkins Interstitial Lung Disease clinic were reviewed for the diagnosis of cryptogenic liver cirrhosis. Two additional patients were identified who underwent liver transplant for decompensated liver cirrhosis and who carried the diagnosis of cryptogenic liver disease (FIG. 9). FIG. 9 shows representative imaging and pathology from patients diagnosed with both idiopathic pulmonary fibrosis and cryptogenic liver cirrhosis. In total, in this series, four of 150 IIP patients were identified with a history of unexplained liver cirrhosis. None of these patients had detectable telomerase mutations although they had telomeres in the lowest percentiles of the population (FIG. 6B and not shown). Based on a prevalence rate of 100/100,000 for both cryptogenic liver cirrhosis and idiopathic pulmonary fibrosis, the likelihood of both diagnoses co-existing in the same individual by chance alone is rare (p<10-22). This association will need to be verified in larger studies. In the meantime, it is intriguing to consider the possibility that telomere shortening, even in the absence of readily detectible mutations, is genetically relevant and underlies an increased predisposition to both pulmonary and liver organ failure that manifest as progressive idiopathic-cryptogenic disease in the same patient.

In the studies presented herein, based on the finding in families with IPF that mutations in telomerase exert their effect through telomere shortening, the incidence of short telomeres in individuals with IIP was examined. Compared to age-matched controls, individuals with IIPs have shorter telomeres than age-matched controls both in peripheral blood and in the lung. Moreover, a subset of patients (10%) with no family history had telomere lengths in the range of mutation carriers even when mutations were not detected. Mutations in hTERT and hTR were readily detectible in only 1% of individuals suggesting that other genetic mechanisms that lead to telomere shortening underlie the prominent differences seen in this cross-sectional study. Even when telomerase is wildtype, telomere-mediated degenerative disease can occur and it is possible that the IPF-IIP phenotype enriches for individuals with the shortest telomeres in the population. These findings support the idea that individuals with the shortest telomeres across the population may be at increased risk for idiopathic interstitial lung disease than individuals with long telomeres and suggest that the genetics of telomere shortening underlie, at least a component of, the age-related predisposition to what appears as unprovoked or idiopathic progressive processes in the lung. This idea is supported by clinical observations of evidence of subclinical IPF in the elderly and suggests that the IPF phenotype may be a clinically important manifestation of aging in the lung. Considering telomere length in future studies examining the risk factors that underlie IPF will be important in fully uncovering its genetic epidemiology.

Approaches to therapy in IPF have been hindered by the escaping pathophysiology that underlies the progressive nature of alveolar destruction and the accumulation of fibrosis. Because of the end replication problem, telomere shortening inevitably occurs in cells over time and short telomeres ultimately activate a DNA damage response that manifests clinically as aplasia in the bone marrow and fibrosis in the lung and liver. As such, it has been proposed that the fibrosis phenotype in parenchymal organs represents an irreversible loss of tissue renewal capacity as a result of the loss of replicative potential of local progenitors (42, 44). Both interstitial lung and liver disease are the most common causes of mortality in dyskeratosis congenita patients who are exposed to cytotoxic chemotherapy in the setting of bone marrow transplant for aplastic anemia (33). Additionally, mice with short telomeres are at increased risk for developing fibrotic liver disease when exposed to toxins compared with wildtype mice (70). The presence of short telomeres in both peripheral blood and the lung of sporadic IPF, beyond a subset of patients who have readily detected telomerase mutations, suggests that telomere shortening plays a role in sporadic disease pathogenesis, and that strategies aimed at preventing cell death or local responses to it, may have an impact in attenuating the course of this disease.

Finally the data presented herein suggest that individuals with IPF may harbor subtle features of a syndrome of telomere shortening. This observation underscores the clinical relevance of short telomeres in the setting of IPF. Idiopathic pulmonary fibrosis and cryptogenic cirrhosis, to our knowledge, have only been described in the setting of dyskeratosis congenita. It will be interesting to examine whether studies of patients with cryptogenic cirrhosis similarly reveal an increased incidence of interstitial lung disease. Systematic studies of personal and family history for aplastic anemia in patients with unexplained fibrosis in the lung or liver will further define the prevalence of a syndrome of telomere shortening.

### Materials and Methods

The invention was performed using, but not limited to, the following materials and methods.

### Subjects

Subjects and their families were recruited into the Vanderbilt Familial Pulmonary Fibrosis Registry on the basis of the presence of two or more cases of idiopathic pulmonary fibrosis. (Families were not limited to those in which only first-degree relatives were affected.) Subjects were excluded from the study if they had a secondary cause of pulmonary fibrosis or if they had skin manifestations suggestive of dyskeratosis congenita. Subjects were recruited from the Vanderbilt Idiopathic Pulmonary Fibrosis Clinic or were referred from other sites in North America between 1996 and 2004. The study was approved by the local institutional review boards, and written informed consent was obtained from all subjects. Diagnostic confirmation was based on a detailed clinical assessment (Table 1, as above, and Table 3, below). Table 3 shows the clinical features of relatives of probands with telomerase mutations.

TABLE 3

| | | Age at | | |
|---|---|---|---|---|
| Pedigree | | diagnosis | | Clinical |
| ID | Gender | (death) | Method of Diagnosis | Impression |
| AII.4 | M | 61 (d.63) | Family history, Autopsy | UIP/IPF |
| BIII.1 | F | 52 (d.63) | Records, CXR, CT, Biopsy | IIP not classifiable |
| BIII.2 | M | 64 (d.67) | Records, CXR, CT, Biopsy | UIP/IPF |
| CI1.1 | M | 67 (d. 68) | Family history | Probable IPF |
| CII.5 | F | 58 (d.61) | Records, CXR, CT | IPF |
| DII.1 | M | 50 (d.56) | Family history, Death certificate | IPF |
| EI.2 | F | 61 (d.64) | Family history, Death certificate | IPF |
| FI.2 | F | (d.69) | Family history | Possible IPF |
| FII.3 | F | (d.70) | Family history | Possible IPF |
| FII.7 | F | 62 (d.63) | Records, CXR, Transbronchial biopsy | IPF |
| FII.9 | F | 54 (d.57) | Family history | Possible IPF |
| FII.11 | M | (d.60) | Records, CXR, Death certificate | IIP not classifiable |
| FIII.2 | M | (d.72) | Family history | Possible IPF |

In Table 2, the abbreviations are as follows: UIP: usual interstitial pneumonia; IIP: idiopathic interstitial pneumonia.

The consensus classification of idiopathic interstitial pneumonia was used in individual cases. At the time the registry was accessed, all 73 probands were reported by their clinicians to be North Americans of European descent.

### Subjects—Sporadic Study

Patients were eligible for the sporadic study if they had a diagnosis of IIP as defined by the 2002 consensus classification (38). Patients were excluded if they had a known family history of IIP at the time of enrollment. Subjects were recruited from the Vanderbilt University Interstitial Lung Disease and the Johns Hopkins Hematology and Interstitial Lung Disease clinics. Patient characteristics are summarized in Table 4, below. The study was approved by the local institutional review boards and written informed consent was obtained from all subjects. Paraffin embedded lung tissue specimens were retrieved on patients with IPF/usual interstitial pneumonia who had a surgical lung biopsy obtained during clinical management. Normal lung tissue was obtained from individuals who died with no recognized lung disorders via the National Disease Research Interchange and the Vanderbilt Autopsy program.

TABLE 4

| | Number |
|---|---|
| | N = 100 |
| Total IIP | 100 |
| Telomere Length studies | 62 |
| Age (years) | |
| Median | 64 |
| Range | 35-80 |
| Sex | |
| Male | 68 |
| Female | 32 |
| IPF | 82 |
| IIP other than IPF | 16 |
| Lung Biopsy | 60 |
| Smoking History | |
| Never | 28 |
| Less than 15 pack-years | 18 |
| 15-50 pack years | 29 |
| ≧50 pack-years | 13 |
| Unknown | 12 |
| Race | |
| Caucasian | 97 |
| Platelet count available (n = 85) | |
| Mean platelet count/mm² | 248 |
| Platelet count less than 150/mm² | 10% |

### Sequence Analysis

Genomic DNA was isolated from peripheral blood with the use of standard methods. hTR was amplified and sequenced in both directions, as described previously (21). The 16 exons of hTERT and its 3′ untranslated region were amplified and sequenced with the use of primers listed in Table 5, shown below.

TABLE 5

| | | | | |
|---|---|---|---|---|
| | Forward | | Reverse | |
| Exon | Primer | Sequence | Primer | Sequence |

| | | | | |
|---|---|---|---|---|
| 1 | TERT1F | gaggtttcaggcagcgctgcgtc | TERT1sR | caggtgaaccagcacgtcgtcgc |
| 1 | TERTsF | caacacggtgaccgacgcactg | TERT1R | cttgtcgcctgaggagtagag |
| 2 | TERT2F | caggacgcgtggaccgagtgacc | TERT2R | gtgaacctcgtaagtttatgc |
| 3 | TERT3F | cttggtgagctggatgtgcggtg | TERT3R | gagccttgcacagaatccacttg |
| 4-5 | TERT4F | gcctcctgaggggctctctattg | TERT5R | ctcccaaggtccagcagggctgc |
| 6 | TERT6F | gccggatccactttcctgactgt | TERT6R | cacagacacgactgcattctagac |
| 7 | TERT7F | gtctgatgtgtggtagctgcag | TERT7R | caacagtctgtccggtcatgag |
| 8 | TERT8F | gtggtcctgcctgtctcagcacc | TERT8R | gcttgccatttccaggcctcgtg |
| 9 | TERT9F | ggctgaatggtagacgtgtcg | TERT9R | gagcagtcatggtctccagagc |
| 10 | TERT10F | gcacagaattgcacaagctgatg | TERT10R | ctgctcttgcggatccagcacc |
| 11 | TERT11F | gcatcctggggctgacattgcc | TERT11R | cagccgggcacaggctccact |
| 12 | TERT12F | gtccatggagtgagcacccagc | TERT12R | cagtcaccatcagccttgcag |
| 13 | TERT13F | gatgacacagagtcttgactcgc | TERT13R | cgcgaacagaactgtgcacag |
| 14 | TERT14F | cacacccacgagcaccgtctg | TERT14R | acagggcgttcaaggatgacc |
| 15 | TERT15F | tgtgactcctcagcctctgtt | TERT15R | cagcagcatctgaggctgctcg |
| 16 | TERT16F | gccgctccagcgtcactg | TERT16R | agtgcacattaggattca |

Amplicons of hTERT were sequenced in one direction, and suspected changes were confirmed in the opposite strand. Mutations in the probands and their relatives were confirmed by bidirectional sequencing. Sequences were inspected manually with the use of Sequencher software, and variants were compared with public databases. Coding and noncoding variants are listed in Table 6, shown below. Table 6 shows the sequence variants in hTERT identified in IPF probands. In Table 6, * rs identifiers refer to base pair changes catalogued in dbSNP v. 126, released in 2006, and ** allele frequency in 528 controls reported in Yamaguchi et al. NEJM 2005.

TABLE 6

| | | | Minor | |
|---|---|---|---|---|
| | | | Allele | |
| | | 3′ flanking | Frequency | Previous |
| Location | Variant | sequence | (per n = 73) | Report* |
| Exon 2 | c.835 G→A (Ala279Thr) | CCGAAGAAGC | 0.03 | 0.02** |
| | c.915 G→A (Ala306Ala) | GGCCCCCCAT | 0.47 | rs2736098 |
| | c.1289 C→T (Ala423Ala) | GGTGTCTGTG | 0.01 | |
| Intron 2 | IVS2 + 39 G→C | CTGAATGCAG | 0.07 | |
| Intron 3 | IVS3 + 130 C→A | GTCACAGGCC | 0.60 | rs7725218 |
| | IVS3 + 137 | GCCTGGTCCA | 0.01 | |
| Exon 4 | c.1812 A→G (Ala604Ala) | GAGGTCAGGC | 0.03 | |
| Intron 4 | IVS4 + 10C→T | TTTGGTTTAA | 0.01 | |
| | IVS4 + 145 A→G | GTGAGGTGGC | 0.42 | rs7734992 |
| | IVS4 + 245 C→T | GGGCCRGGGC | 0.26 | rs2242652 |
| | IVS4 + 309 C→G | GGGCCRGGGC | 0.38 | rs10054203 |
| | IVS4 + 483 G→A | TGGCATGAGG | 0.29 | rs10069690 |
| | IVS4 + 572 G→A | GGTCTGGGTG | 0.01 | |
| Exon 5 | c.2031 C→T (Gly677Gly) | GCCTCTGTGC | 0.01 | |
| | Ala699Ala C→T | GCCGCTGTAC | 0.03 | |
| | c.2031 G→A (Pro702Pro) | CCGCCTGAGC | 0.01 | |
| Intron 5 | IVS5 + 178 A→C | TGGGGCCGAC | 0.10 | |
| Intron 6 | IVS6 + 180 G→A | CCCCCGTTTC | 0.03 | |
| | IVS6-156_−155 ins. | GTTTCCCAAA | 0.01 | |
| | GGCCCCC2→GGCCCCC3 | | | |
| Intron 9 | IVS9 + 121 C > T | GCTTTGGAGA | 0.01 | |
| Intron 12 | IVS12 + 25-27 + AG→AG | GTTCAGAGTT | 0.01 | |
| | IVS12 + 59 TG8→TG9 | CRCGCGTGCC | 0.01 | |
| | IVS12 + 75 G→A | CGCGTGCCTG | 0.04 | |
| Intron 13 | IVS13 + 46 C→T | GCGCTCTGCT | 0.01 | |
| | IVS13 + 137 C→A | CTGTGCACAG | 0.12 | |
| Exon 14 | c.3039 C→T (His1013His) | GCATTGTGC | 0.14 | |
| Intron 14 | IVS14 + 85 C→T | CCCGCAGACC | 0.01 | |
| Exon 15 | c.3184 G→A (Ala1062Thr) | CCGGCCCTST | 0.07 | 0.01** |
| | c.3193 C→G (Leu1065Leu) | TGCCCTCCGA | 0.01 | |
| Intron 15 | IVS15 101 C→T | GCCCCCGGGC | 0.01 | |
| Exon 16 | c.3324 G→A (Pro1108Pro) | GGAAGCTCCC | 0.05 | |
| 3'UTR | C*99 C→T | GCACCGCTGGG | 0.49 | rs2853890 |

### Telomeres and Telomerase

A reverse-transcriptase polymerase-chain-reaction (RT-PCR) assay was performed with the use of RNA isolated from peripheral blood to make complementary DNA (cDNA). Primers were designed to span exons where a mutation was predicted to alter splicing. PCR products were cloned, and the sequence was verified.

The average length of telomeres was measured in peripheral-blood lymphocytes by flow fluorescence in situ hybridization (FISH), as described previously (28).

In certain examples, point mutations were generated, and the telomerase complex was reconstituted in vitro (24). Telomerase activity was assayed without amplification, with the use of a modified direct assay (29,30).

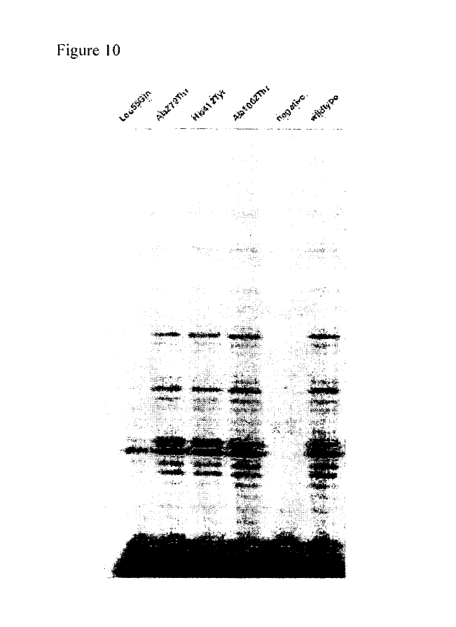

In certain examples, telomere length was measured in paraffin-embedded tissues in alveolar type 2 cells using quantitative FISH as described. Quantitation of telomere length was specific to surfactant protein C positive cells identified by immunostaining with rabbit anti-human SPC antibodies (Chemicon, Calif. USA) followed by detection with goat anti-Rabbit Alexa Fluor-488 conjugated antibody (Invitrogen, Oreg. USA). Telomere length was measured in each cell by dividing the total Cy3 signal (telomere signal) in the nucleus by the total DNA signal as quantitated by the DAPI intensity. 15 nuclei were analyzed for each sample using Telometer, an ImageJ plugin available at http://bui2.win.ad.jhu.edu/telometer/. hTERT and hTR were sequenced from genomic DNA prepared from peripheral blood as described (42). hTERT variants are listed in Table 7, below. Table 7 shows hTERT variants identified in sporadic IIP patients. In Table 7, * rs identifiers refer to dbSNP cluster id; v.128; ** Allele frequency in 528 controls as reported in Yamaguchi et al NEJM 2005. † The functional significance of these non-synonymous variants was examined in the telomerase activity assays shown in FIG. 10.

TABLE 7

| | | | Minor | |
|---|---|---|---|---|
| | | | Allele | |
| | | 3′ flanking | Frequency | Previous |
| Location | Variation | sequence | (n = 73) | report* |
| [symbol] | [symbol] | [symbol] | [symbol] | [symbol] |

[symbol]

indicates data missing or illegible when filed

To assess the functional significance of suspected mutants, point mutations were generated, and the telomerase complex was reconstituted in vitro (42, 44, 64, 64a) and in VA13 cells that lacked telomerase as modified from (65). The direct assay and activity was quantitated based on at least three independent experiments. Statistical analyses were performed using Stata 10.0 for Windows (Stata Corporation, Tex. USA). All p-values are 2-sided and error bars represent standard error of the mean. All the analyses were performed blind (65). Telomerase activity was assayed without amplification with the use of the direct assay and activity was quantitated based on at least three independent experiments. Statistical analyses were performed using Stata 10.0 for Windows (Stata Corporation, Tex. USA). All p-values are 2-sided and error bars represent standard error of the mean. All the analyses were performed blind.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. Lawson W E, Loyd J E. The genetic approach in pulmonary fibrosis: can it provide clues to this complex disease? Proc Am Thorac Soc 2006; 3:345-9.
2. Loyd J E. Pulmonary fibrosis in families. Am J Respir Cell Mol Biol 2003; 29: Suppl:S47-550.
3. Marshall R P, Puddicombe A, Cookson W O, Laurent G J. Adult familial cryptogenic fibrosing alveolitis in the United Kingdom. Thorax 2000; 55:143-6.
4. Thomas A Q, Lane K, Phillips J III, et al. Heterozygosity for a surfactant protein C gene mutation associated with usual interstitial pneumonitis and cellular nonspecific interstitial pneumonitis in one kindred. Am J Respir Crit Care Med 2002; 165:1322-8.
5. Greider C W, Blackburn E H. Identification of a specific telomere terminal transferase activity in Tetrahymena extracts. Cell 1985; 43:405-13.
6. Idem. A telomeric sequence in the RNA of Tetrahymena telomerase required for telomere repeat synthesis. Nature 1989; 337:331-7.
7. Nakamura T M, Morin G B, Chapman K B, et al. Telomerase catalytic subunit homologs from fission yeast and human. Science 1997; 277:955-9.
8. Lingner J, Hughes T R, Shevchenko A, Mann M, Lundblad V, Cech T R. Reverse transcriptase motifs in the catalytic subunit of telomerase. Science 1997; 276:561-7.
9. Harley C B, Futcher A B, Greider C W. Telomeres shorten during ageing of human fibroblasts. Nature 1990; 345:458-60.
10. Vaziri H, Dragowska W, Allsopp R C, Thomas T E, Harley C B, Lansdorp P M. Evidence for a mitotic clock in human hematopoietic stem cells: loss of telomeric DNA with age. Proc Natl Acad Sci U S A 1994; 91:9857-60.
11. Lee H W, Blasco M A, Gottlieb G J, Homer J W II, Greider C W, DePinho R A. Essential role of mouse telomerase in highly proliferative organs. Nature 1998; 392:569-74.
12. d'Adda di Fagagna F, Reaper P M, Clay Farrace L, et al. A DNA damage checkpoint response in telomere-initiated senescence. Nature 2003; 426:194-8.
13. Hemann M T, Strong M A, Hao L Y, Greider C W. The shortest telomere, not average telomere length, is critical for cell viability and chromosome stability. Cell 2001; 107:67-77.
14. Hao L Y, Armanios M, Strong M A, et al. Short telomeres, even in the presence of telomerase, limit tissue renewal capacity. Cell 2005; 123:1121-31.
15. Rudolph K L, Chang S, Lee H W, et al. Longevity, stress response, and cancer in aging telomerase-deficient mice. Cell 1999; 96:701-12.
16. Dokal I, Vulliamy T. Dyskeratosis congenita: its link to telomerase and aplastic anaemia. Blood Rev 2003; 17:217-25.
17. Dokal I. Dyskeratosis congenita in all its forms. Br J Haematol 2000; 110:768-79.
18. Knight S, Vulliamy T, Copplestone A, Gluckman E, Mason P, Dokal I. Dyskeratosis Congenita (DC) Registry: identification of new features of DC. Br J Haematol 1998; 103:990-6.
19. Heiss N S, Knight S W, Vulliamy T J, et al. X-linked dyskeratosis congenita is caused by mutations in a highly conserved gene with putative nucleolar functions. Nat Genet 1998; 19:32-8.
20. Fogarty P F, Yamaguchi H, Wiestner A, et al. Late presentation of dyskeratosis congenita as apparently acquired aplastic anaemia due to mutations in telomerase RNA. Lancet 2003; 362:1628-30.
21. Vulliamy T, Marrone A, Goldman F, et al. The RNA component of telomerase is mutated in autosomal dominant dyskeratosis congenita. Nature 2001; 413:432-5.
22. Yamaguchi H, Baerlocher G M, Lansdorp P M, et al. Mutations of the human telomerase RNA gene (TERC) in aplastic anemia and myelodysplastic syndrome. Blood 2003; 102:916-8.
23. Yamaguchi H, Calado R T, Ly H, et al. Mutations in TERT, the gene for telomerase reverse transcriptase, in aplastic anemia. N Engl J Med 2005; 352:1413-24.
24. Armanios M, Chen J L, Chang Y P, et al. Haploinsufficiency of telomerase reverse transcriptase leads to anticipation in autosomal dominant dyskeratosis congenita. Proc Natl Acad Sci USA 2005; 102:15960-4.
25. Vulliamy T, Marrone A, Szydlo R, Walne A, Mason P J, Dokal I. Disease anticipation is associated with progressive telomere shortening in families with dyskeratosis congenita due to mutations in TERC. Nat Genet 2004; 36:447-9.
26. Blasco M A, Lee H W, Hande M P, et al. Telomere shortening and tumor formation by mouse cells lacking telomerase RNA. Cell 1997; 91:25-34.
27. American Thoracic Society/European Respiratory Society International Multi-disciplinary Consensus Classification of the Idiopathic Interstitial Pneumonias: this joint statement of the American Thoracic Society (ATS), and the European Respiratory Society (ERS) was adopted by the ATS board of directors, June 2001 and by the ERS Executive Committee, June 2001. Am J Respir Crit Care Med 2002; 165:277-304. [Erratum, Am J Respir Crit Care Med 2002; 166:426.]
28. Baerlocher G M, Vulto V, de Jong G, Lansdorp P M. Flow cytometry and FISH to measure the average length of telo-meres (flow FISH). Nat Protoc (in press).
29. Chen J L, Greider C W. Template boundary definition in mammalian telomerase. Genes Dev 2003; 17:2747-52.
30. Drosopoulos W C, Direnzo R, Prasad V R. Human telomerase RNA template sequence is a determinant of telomere repeat extension rate. J Biol Chem 2005; 280: 32801-10.
31. National Institute of Environmental Health Sciences. TERT: telomerase reverse transcriptase. (Accessed Mar. 5, 2007, at http://egp.gs.washington.eduldatalterti.)
32. Jacobs S A, Podell E R, Cech T R. Crystal structure of the essential N-terminal domain of telomerase reverse transcriptase. Nat Struct Mol Biol 2006; 13:218-25.
33. Autexier C, Lue N F. The structure and function of telomerase reverse transcriptase. Annu Rev Biochem 2006; 75: 493-517.
34. Chen J L, Blasco M A, Greider C W. Secondary structure of vertebrate telomerase RNA. Cell 2000; 100:503-14.
35. Steele M P, Speer M C, Loyd J E, et al. Clinical and pathologic features of familial interstitial pneumonia. Am J Respir Crit Care Med 2005; 172:1146-52.
36. Valdes A M, Andrew T, Gardner J P, et al. Obesity, cigarette smoking, and telomere length in women. Lancet 2005; 366: 662-4.
37. Tsuji T, Aoshiba K, Nagai A. Alveolar cell senescence in patients with pulmonary emphysema. Am J Respir Crit Care Med 2006; 174:886-93.
38. American Thoracic Society/European Respiratory Society International Multidisciplinary Consensus Classification of the Idiopathic Interstitial Pneumonias. This joint statement of the American Thoracic Society (ATS), and the European Respiratory Society (ERS) was adopted by the ATS board of directors, June 2001 and by the ERS Executive Committee, June 2001. Am J Respir Crit Care Med 2002; 165(2):277-304.
39. Loyd J E Pulmonary fibrosis in families. Am J Respir Cell Mol Biol 2003; 29(3 Suppl):547-50.
40. Steele M P, Speer M C, Loyd J E, et al. Clinical and pathologic features of familial interstitial pneumonia. Am J Respir Crit Care Med 2005; 172(9):1146-52.
41. Thomas A Q, Lane K, Phillips J, 3rd, et al. Heterozygosity for a surfactant protein C gene mutation associated with usual interstitial pneumonitis and cellular nonspecific interstitial pneumonitis in one kindred. Am J Respir Crit Care Med 2002; 165(9):1322-8.
42. Armanios M Y, Chen J J, Cogan J D, et al. Telomerase mutations in families with idiopathic pulmonary fibrosis. N Engl J Med 2007; 356(13):1317-26.
43. Tsakiri K D, Cronkhite J T, Kuan P J, et al. Adult-onset pulmonary fibrosis caused by mutations in telomerase. Proc Natl Acad Sci U S A 2007; 104(18):7552-7.
44. Armanios M, Chen J L, Chang Y P, et al. Haploinsufficiency of telomerase reverse transcriptase leads to anticipation in autosomal dominant dyskeratosis congenita. Proc Natl Acad Sci U S A 2005; 102(44):15960-4.
45. Blasco M A, Lee H W, Hande M P, et al. Telomere shortening and tumor formation by mouse cells lacking telomerase RNA. Cell 1997; 91(1):25-34.
46. Hao L Y, Armanios M, Strong M A, et al. Short telomeres, even in the presence of telomerase, limit tissue renewal capacity. Cell 2005; 123(6):1121-31.
47. Vulliamy T, Marrone A, Szydlo R, Walne A, Mason P J, Dokal I. Disease anticipation is associated with progressive telomere shortening in families with dyskeratosis congenita due to mutations in TERC. Nat Genet 2004.
48. d'Adda di Fagagna F, Reaper P M, Clay-Farrace L, et al. A DNA damage checkpoint response in telomere-initiated senescence. Nature 2003; 426(6963):194-8.
49. Harley C B, Futcher A B, Greider C W. Telomeres shorten during ageing of human fibroblasts. Nature 1990; 345(6274):458-60.
50. Lee H W, Blasco M A, Gottlieb G J, Homer J W, 2nd, Greider C W, DePinho R A. Essential role of mouse telomerase in highly proliferative organs. Nature 1998; 392(6676):569-74.
51. Greider C W, Blackburn E H. Identification of a specific telomere terminal transferase activity in Tetrahymena extracts. Cell 1985; 43(2 Pt 1):405-13.
52. Greider C W, Blackburn E H. The telomere terminal transferase of Tetrahymena is a ribonucleoprotein enzyme with two kinds of primer specificity. Cell 1987; 51(6):887-98.
53. Lingner J, Hughes T R, Shevchenko A, Mann M, Lundblad V, Cech T R. Reverse transcriptase motifs in the catalytic subunit of telomerase. Science 1997; 276(5312):561-7.
54. Nakamura T M, Morin G B, Chapman K B, et al. Telomerase catalytic subunit homologs from fission yeast and human. Science 1997; 277(5328):955-9.
55. Vulliamy T, Marrone A, Goldman F, et al. The RNA component of telomerase is mutated in autosomal dominant dyskeratosis congenita. Nature 2001; 413(6854):432-5.
56. Yamaguchi H, Calado R T, Ly H, et al. Mutations in TERT, the gene for telomerase reverse transcriptase, in aplastic anemia. N Engl J Med 2005; 352(14):1413-24.
57. Dokal I. Dyskeratosis congenita in all its forms. Br J Haematol 2000; 110(4):768-79.
58. Dokal I. Dyskeratosis congenita. A disease of premature ageing. Lancet 2001; 358 Suppl:S27.
59. Heiss N S, Knight S W, Vulliamy T J, et al. X-linked dyskeratosis congenita is caused by mutations in a highly conserved gene with putative nucleolar functions. Nat Genet 1998; 19(1):32-8.
60. Mitchell J R, Wood E, Collins K. A telomerase component is defective in the human disease dyskeratosis congenita. Nature 1999; 402(6761):551-5.
61. Alter B P, Baerlocher G M, Savage S A, et al. Very short telomere length by flow fluorescence in situ hybridization identifies patients with dyskeratosis congenita. Blood 2007; 110(5):1439-47.
62. Baerlocher G M, Vulto I, de Jong G, Lansdorp PM. Flow cytometry and FISH to measure the average length of telomeres (flow FISH). Nat Protoc 2006; 1(5):2365-76.
63. Meeker A K, Gage W R, Hicks J L, et al. Telomere length assessment in human archival tissues: combined telomere fluorescence in situ hybridization and immunostaining. Am J Pathol 2002; 160(4):1259-68.
64. Chen J L, Greider C W. Template boundary definition in mammalian telomerase. Genes Dev 2003; 17(22):2747-52.
64a. Drosopoulos W C, Direnzo R, Prasad V R. Human telomerase RNA template sequence is a determinant of telomere repeat extension rate. J Biol Chem 2005; 280(38):32801-10.
65. Cristofari G, Lingner J. Telomere length homeostasis requires that telomerase levels are limiting. Embo J 2006; 25(3):565-74.
66. Chen J L, Blasco M A, Greider C W. Secondary structure of vertebrate telomerase RNA. Cell 2000; 100(5):503-14.
67. Yamaguchi H, Baerlocher G M, Lansdorp P M, et al. Mutations of the human telomerase RNA gene (TERC) in aplastic anemia and myelodysplastic syndrome. Blood 2003; 102(3):916-8.
68. SNPs NIoEHSEGPN.
69. de la Fuente J, Dokal I. Dyskeratosis congenita: advances in the understanding of the telomerase defect and the role of stem cell transplantation. Pediatr Transplant 2007; 11(6):584-94.
70. Rudolph K L, Chang S, Millard M, Schreiber-Agus N, DePinho R A Inhibition of experimental liver cirrhosis in mice by telomerase gene delivery. Science 2000; 287(5456):1253-8.

1. A method of diagnosing a telomere-associated disease or disorder in a subject, comprising:
providing a sample from the subject; and
analyzing all or part of a nucleic acid sequence corresponding to one or more of the telomere genes in the sample for the presence of one or more alterations, wherein the presence of at least one alteration is indicative of an increased risk of the presence of a telomere-associated disease or disorder.

2. The method of claim 1, wherein the telomere associated gene is selected from hTERT (telomerase reverse transcriptase) and hTR (telomerase RNA).

3. The method of claim 1, further comprising the step of determining the average telomere length.

4. A method of diagnosing a telomere-associated disease or disorder in a subject, comprising:
providing a sample from a the subject; and
determining the average telomere length in the sample compared to a control, wherein a shorter average telomere length is indicative of an increased risk of the presence of a telomere-associated disease or disorder.

5. The method according to claim 1, wherein the telomere-associated disease or disorder is selected from the group consisting of: dyskeratosis congenita, hematopoietic defects, idiopathic pulmonary fibrosis, and cryptogenic liver cirrhosis.

6-9. (canceled)

10. The method according to claim 1, wherein the one or more alterations is selected from the group consisting of: SEQ ID NO: 1, SEQ ID NO: 2, SEQ ID NO: 3, SEQ ID NO: 4, SEQ ID NO: 5, and SEQ ID NO: 6.

11. (canceled)

12. The method according to claim 1, wherein the analysis further comprises an amplification step.

13. The method according to claim 1, wherein the analysis further comprises a hybridization step using at least one primer set specific for the sequence to identify sequence alterations.

14. The method of claim 13, wherein the primer set comprises a forward primer selected from the group consisting of SEQ ID NOs: 11-27 and a reverse primer selected from the group consisting of SEQ ID NOs: 28-42.

15-19. (canceled)

20. A method of diagnosing a presence or a predisposition to a telomere-associated disease or disorder in a subject, comprising:
providing a sample from the subject; and
analyzing all or part of a nucleic acid sequence corresponding to one or more telomere genes in the sample for the presence of one or more alterations in the telomere genes, wherein the analyzing comprises amplifying all or part of the nucleic acid sequence of the telomere genes prior to detection, said amplifying being performed with at least one primer consisting of the sequence of any one of SEQ ID NOs: 11-42, wherein the presence of at least one alteration is indicative of an increased risk for the presence or predisposition to a telomere-associated disease or disorder.

21. A method of detecting the presence or absence of an alteration in a nucleic acid in a sample, comprising:
analyzing all or part of a nucleic acid corresponding to a telomere associated gene in the sample and a control sample to determine whether one or more alterations are present in the sample nucleic acid; and
determining the presence or absence of one or more alterations in the sample nucleic acid compared to the control sample nucleic acid.

22-23. (canceled)

24. A method of diagnosing of diagnosing a presence or a predisposition to a telomere-associated disease or disorder in a subject, comprising:
providing a sample from a the subject; and
determining the average length of telomeres in the sample compared to a control, wherein a shorter average telomere length is indicative of an increased risk for the presence or predisposition to a telomere-associated disease or disorder.

25-58. (canceled)

59. An oligonucleotide primer for detecting in a subject a genetic risk for developing a telomere-associated disease or disorder comprising the sequence selected from the group consisting of: SEQ ID NOs: 11-42, or
a primer set for detecting in a subject a genetic risk for developing a telomere-associated disease or disorder, the primer set having a forward primer selected from the group consisting of: SEQ ID NOs: 11-27 and a reverse primer selected from the group consisting of: SEQ ID NOs: 28-42, or
a kit for use in diagnosing a telomere-associated disease or disorder in a subject or diagnosing the presence or a predisposition a telomere-associated disease or disorder in a subject, and instructions for use.

60-62. (canceled)
